(19) United States
(12) Patent Application Publication
(10) Pub. No.: US 2024/0112787 A1
(43) Pub. Date: Apr. 4, 2024

(54) SYSTEMS AND METHODS FOR MANAGING BENEFICIAL PRODUCTS

(71) Applicant: VendRx, Inc., South Jordan, UT (US)

(72) Inventors: Trent Jefferies, South Jordan, UT (US); Russell McKee, Santa Monica, CA (US); Doug Wozny, Norco, CA (US)

(73) Assignee: VendRx, Inc., South Jordan, UT (US)

(21) Appl. No.: 18/312,373

(22) Filed: May 4, 2023

Related U.S. Application Data
(60) Provisional application No. 63/339,328, filed on May 6, 2022.

(51) Int. Cl. G16H 40/20; G07C 9/00

(52) U.S. Cl. CPC G16H 40/20; G07C 9/00896; G06K 7/1417

(57) ABSTRACT

Vendable units (VU) are maintained within respective compartments of a dispensing apparatus. The VU are managed in accordance with a hierarchical identifier (ID) scheme. VU are assigned a first identifier determinative of the type of beneficial product contained within the VU and a second identifier determinative of the lot or batch of the beneficial product. VU are assigned to compartments based on a combination of the first and second ID. During operations to load VU into the apparatus, the compartment assigned to the VU is unlocked while other compartments are locked, reducing the potential for human error. Similarly, when dispensing VU from the apparatus, the compartment comprising the VU to be dispensed to the patient is unlocked while other compartments remain locked, further reducing the potential for error.

### CROSS-REFERENCE TO RELATED APPLICATIONS

This application claims priority to U.S. Provisional Patent Application No. 63/339,328 entitled SYSTEMS AND METHODS FOR MANAGING BENEFICIAL PRODUCTS, which was filed May 6, 2022, and which is hereby incorporated by reference.

### BACKGROUND

A health care provider (HCP) can provide beneficial products directly to patients. This type of “direct dispensing” model is often more convenient for patients, since they can obtain products, such as prescription drugs, directly on site without traveling to a separate location, such as a pharmacy or the like. Direct dispensing models can also provide an additional revenue stream for the HCP.

Managing the distribution of beneficial products, however, can involve significant overhead. In direct dispensing models, the HCP may have the responsibility for regulatory compliance, adjudication, coordination of benefits, accounting, and so on. Tasks related to receiving, storing, securing, and tracking drugs dispensed to patients are often highly regulated. For example, the HCP may be required to track the types of medications dispensed to respective patients. The HCP may also be required to track more granular information, such as lot or batch identifiers so that, inter alia, patients can be notified of recalls and/or the like. As such, although direct dispensing models can be beneficial for both patients and HCP, they are often not feasible, particularly in low-volume scenarios. Therefore, what is needed are systems and methods to efficiently manage the intake, management, distribution, and tracking of beneficial products in direct dispensing and other health care models.

### BRIEF DESCRIPTION OF THE DRAWINGS

Examples of systems, methods, devices, and computer-readable storage media comprising instructions configured to efficiently manage the intake, management, distribution, and tracking of beneficial products.

FIG. 1A is a schematic block diagram illustrating an operating environment comprising a system for managing the distribution of beneficial products.

FIG. 1B illustrates an example of a dispensable unit.

FIGS. 1C-1D illustrate examples of dispensing apparatus compartments.

FIGS. 1E-1F illustrate further examples of dispensing apparatus compartments.

FIG. 2A illustrates an example of a modular dispensing apparatus.

FIG. 2B illustrates examples of compartments having different internal configurations.

FIG. 3 illustrates another example of a modular dispensing apparatus.

FIG. 4A is a schematic block diagram illustrating another example of a dispensing apparatus.

FIG. 4B is a schematic block diagram illustrating an example of a system configured to fulfill aspects of a provisioning operation.

FIG. 5A is a schematic block diagram illustrating an example of a data schema for maintaining information pertaining to the distribution of beneficial products within a computer-readable data store.

FIG. 5B is a schematic block diagram illustrating an example of a data schema for auditing operations pertaining to the management of beneficial products.

FIG. 6A is a flow diagram illustrating an example of a method for managing intake operations to load beneficial products into a dispensing apparatus.

FIG. 6B is a flow diagram illustrating an example of a method for selecting a dispensing apparatus compartment for an intake operation.

FIG. 6C is a flow diagram illustrating another example of a method for managing intake operations.

FIG. 6D is a flow diagram illustrating further examples of methods for managing intake operations.

FIG. 7A is a flow diagram illustrating an example of a method for managing fulfilment operations to dispense beneficial products from a dispensing apparatus.

FIG. 7B is a flow diagram illustrating an example of a method for selecting beneficial products to dispense in a fulfilment operation.

FIG. 7C is a flow diagram illustrating another example of a method for managing fulfilment operations to dispense beneficial products from a dispensing apparatus.

FIG. 7D is a flow diagram illustrating further examples of methods for managing fulfilment operations to dispense beneficial products from a dispensing apparatus.

FIG. 8 is a flow diagram illustrating an example of a method for fulfilling prescriptions issued to patients.

FIG. 9A is a flow diagram illustrating another example of a method for managing beneficial products.

FIG. 9B is a flow diagram illustrating an example of a method for assigning a compartment to a vendable unit.

FIG. 9C is a flow diagram illustrating another example of a method for assigning a compartment to a vendable unit.

FIG. 9D is a flow diagram illustrating an example of a method for selecting a compartment to service a fulfillment operation.

FIG. 10 is a flow diagram illustrating an example of a method for handling events pertaining to beneficial products.

### DETAILED DESCRIPTION

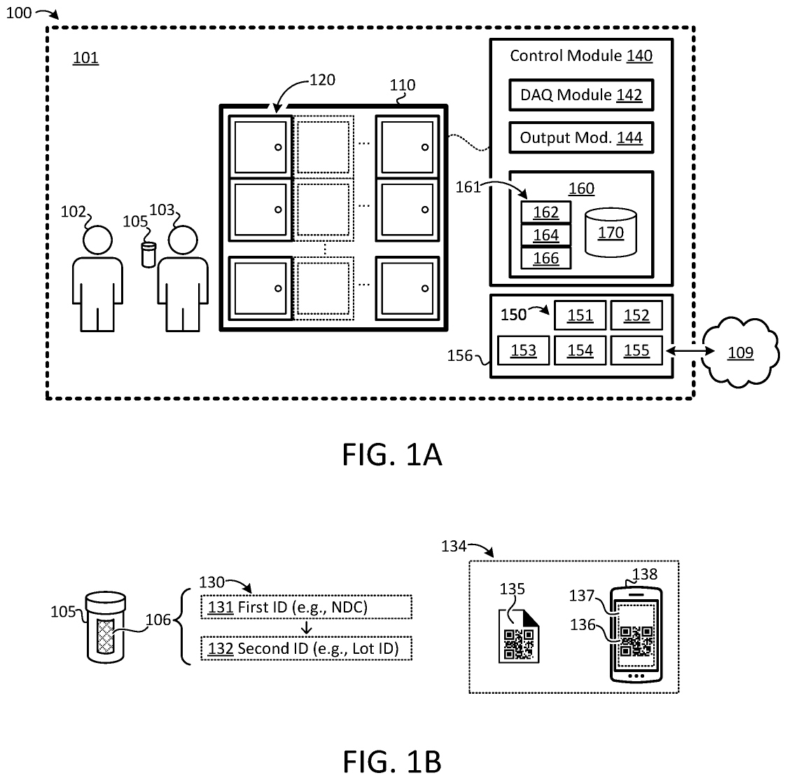

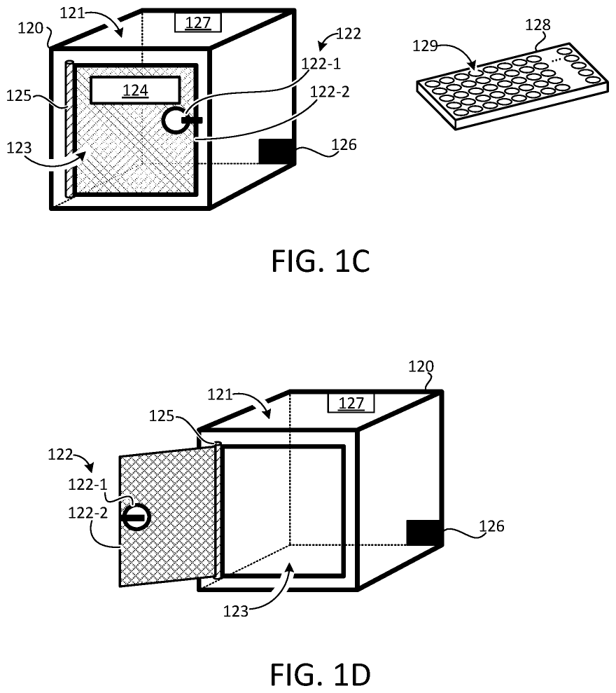

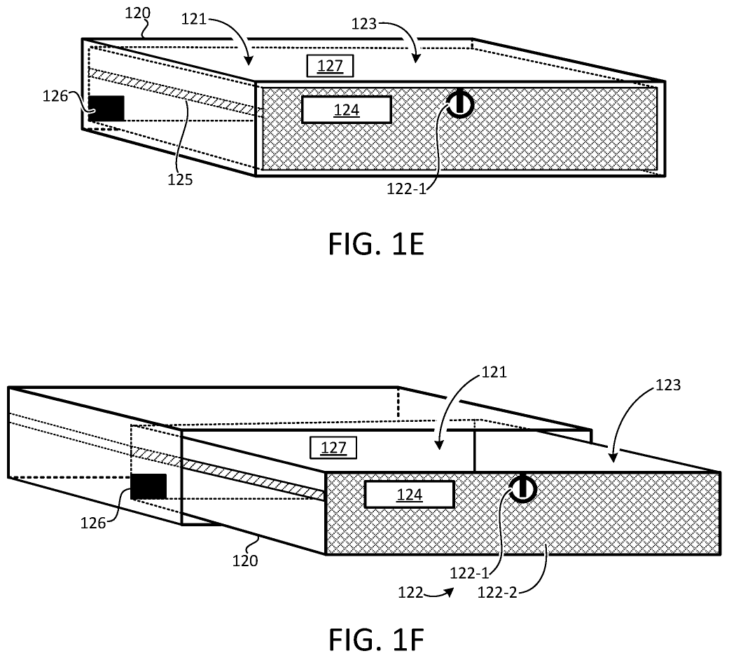

FIG. 1A is a schematic block diagram illustrating an example of an operating environment comprising a system 100 for managing the distribution of beneficial products. The system 100 may be utilized to implement a direct dispensing model in which an HCP 101 dispenses beneficial products to patients 102. In some implementations, the beneficial products may be dispensed directly to patients 102. Alternatively, or in addition, Alternatively, or in addition, personnel 103 of the HCP 101 may facilitate direct dispensing to patients 102, as illustrated in FIG. 1A. The disclosure is not limited in this regard, however, and may be adapted for any suitable distribution model.

As used herein, an HCP 101 may comprise and/or refer to any person, entity, and/or organization that provides health care services, including, but not limited to: a doctor, a doctor's office, a dentist, a dentist's office, an optometrist, an optometrist's office, an ophthalmologist, an ophthalmologist's office, a physical therapist, a physical therapist's office, a veterinarian, a veterinarian's office, a clinic, a primary care clinic, a specialized clinic (e.g., a clinic specializing in cardiology, dentistry, dermatology, ear, nose, and throat (ENT), gastroenterology, gynecology, obstetrics, neurology, nutrition, oncology, ophthalmology, orthopedics, podiatry, physical therapy, sports medicine, urology, and/or the like), a sexual health clinic, a mental health clinic, an addiction services clinic, a community health center, a retail clinic (e.g., a clinic at a retail location and/or located within a retail store), a rural health clinic, a dialysis clinic, a mobile clinic, an urgent care clinic, a hospital, an emergency room, and/or the like.

The system 100 may comprise an apparatus 110 for dispensing beneficial products (an apparatus 110 or dispensing apparatus 110). The HCP 101 may utilize the apparatus 110 to, inter alia, control the intake, management, distribution, and tracking of beneficial products. The beneficial products may be maintained within compartments 120 of the apparatus 110. The beneficial products may be tracked in accordance with a hierarchical identifier (ID) scheme 130. As disclosed in further detail herein, the hierarchical ID scheme 130 may be used to group beneficial products within compartments 120 of the apparatus 110 by both a) type and b) lot or batch. Grouping beneficial products in this manner may help reduce the potential for error. More specifically, using multiple ID to group medications within respective access-controlled compartments 120 may ensure that medication is accurately dispensed to patients 102, e.g., ensure that patients 102 receive not only the correct type of medication, but also medication from the correct lot or batch.

The dispensing apparatus 110 may be configured for use with any suitable type of beneficial products, including, but not limited to: controlled substances, pharmaceuticals, pharmaceutical preparations, drugs, prescription drugs, pills, drops, suppositories, patches, powders, ointments, sprays, injections, consumables, and/or the like. The dispensing apparatus 110 may comprise any suitable dispensing means, including, but not limited to: a dispensing machine, a dispensing station, a dispensing appliance, a dispensing mechanism, and/or the like.

As illustrated in FIG. 1B, beneficial products may be provisioned by and/or within vendable units 105. As used herein, a vendable unit (VU) 105, may comprise and/or refer to any suitable means for vending, dispensing, packaging and/or otherwise provisioning a beneficial product to a patient 102 including, but not limited to: a container, a tamper resistant container, a bag, a tube, a bottle, a pill bottle, a dropper bottle, a prescription bottle, a spray bottle, a blister pack, a box, a packet, a vial, a kit, a nutrition kit, an inhaler, an autoinjector, and/or the like.

In some implementations, VU 105 may comprise and/or be associated with respective data tags 106. As used herein, a data tag 106 (or DT 106) may comprise any suitable means for representing, encoding, storing, and/or otherwise providing information associated with a VU 105. A DT 106 may include, but us not limited to: a label, text, an identifier (ID), text configured for optical character recognition (OCR), marks, marks configured to optical mark recognition (OMR), a code, an optical code, a barcode, a barcode configured for optical barcode recognition (OBR), a linear or one-dimensional (1D) barcode, a two-dimensional (2D) barcode, a matrix code, a matrix barcode, a quick response (QR) code, a Bokode, a multi-row barcode, a color barcode, an electromagnetic label, a magnetic code, a magnetic stripe, a smart card, a chip, an electromagnetic data tag, a radio-frequency identification (RFID) tag, and/or the like. The DT 106 of a VU 105 may comprise any suitable information pertaining to the VU 105 and/or beneficial product(s) of the VU 105, e.g., may comprise an NDC, lot ID, Universal Product Code (UPC), RxNorm, Global Trade Identification Number (GTIN), and/or the like.

Referring to FIG. 1A, the dispensing apparatus 110 may further comprise a controller or control module 140. The control module 140 may comprise and/or be implemented by computing resources 150. As used herein, computing resources 150 may comprise any suitable computing means including, but not limited to processing resources 151, memory resources 152, non-transitory (NT) storage resources 153, human-machine interface (HMI) resources 154, a data interface 155, and/or the like. The processing resources 151 may comprise any suitable processing means including, but not limited to: processing circuitry, logic circuitry, an integrated circuit (IC), a processor, a processing unit, a physical processor, a virtual processor (e.g., a virtual machine), an arithmetic-logic unit (ALU), a central processing unit (CPU), a general-purpose processor, a programmable logic device (PLD), a field programmable gate array (FPGA), an application-specific integrated circuit (ASIC), a System on Chip (SoC), virtual processing resources, and/or the like.

The memory resources 152 may comprise any suitable memory means including, but not limited to: volatile memory, non-volatile memory, random access memory (RAM), dynamic RAM (DRAM), synchronous DRAM (SDRAM), cache memory, or the like. The NT storage resources 153 may comprise any suitable non-transitory, persistent, and/or non-volatile storage means including, but not limited to: a non-transitory storage device, a persistent storage device, an internal storage device, an external storage device, a remote storage device, Network Attached Storage (NAS) resources, a magnetic disk drive, a hard disk drive (HDD), a solid-state storage device (SSD), a Flash memory device, and/or the like.

The HMI resources 154 may comprise any suitable means for human-machine interaction including, but not limited to: an input device, an output device, an input/output (I/O) device, a visual output device, a light emitting diode (LED), a display device, a monitor, a touch screen, a keyboard, a gesture input device, a mouse, a haptic feedback device, an audio output device, a neural interface device, and/or the like.

The data interface 155 may comprise any suitable data communication and/or interface means including, but not limited to: a communication interface, a I/O interface, a network interface, an electronic communication network interface, an interconnect, and/or the like. The data interface 155 may be configured for electronic communication via a network 109, which may include, but is not limited to one or more of an Internet Protocol (IP) network, a wired network, a wireless network (e.g., IEEE 802.11a-n wireless network, Bluetooth® network, Near-Field Communication (NFC) network, and/or the like), a Local Area Network (LAN), a Wide Area Network (WAN), a Virtual Private Network (VPN), and/or the like. The data interface 155 may be further configured to communicatively and/or operatively couple the control module 140 to respective compartments 120 of the dispensing apparatus 110, as disclosed in further detail herein.

The computing resources 150 of the control module 140 may be implemented and/or embodied by the dispensing apparatus 110 and/or another electronic device 156. The electronic device 156 may comprise any suitable computing resource means, including, but not limited to: a computing device, a portable computing device, a tablet computer, a smart phone, a personal digital assistant, a terminal, and/or the like.

The control module 140 may comprise and/or be coupled to a data acquisition (DAQ) module 142 and an output module 144. The DAQ module 142 may comprise any suitable means for interrogating VU 105. As used herein, interrogating a VU 105 (or an interrogation operation) refers to an operation to read, sense, interpret, extract, derive, and/or otherwise acquire data pertaining to a VU 105. In other words, interrogating a VU 105 may comprise reading data encoded on or within a DT 106 of the VU 105. As disclosed in further detail herein, interrogating a VU 105 may comprise retrieving a plurality of ID of the VU 105, such as a first ID 131 and a second ID 132 of a hierarchical ID scheme 130). The DAQ module 142 may implement interrogation operations by use of one or more acquisition devices. The DAQ module 142 may comprise and/or be coupled to any suitable means for retrieving data from a VU 105 (and/or DT 106 thereof), which may include, but is not limited to an optical scanner, an optical reader, a barcode reader, an OCR device, an OMR device, a computer vision device, an RFID device, an RFID reader, an NFC device, and/or the like.

In some implementations, interrogating a VU 105 may comprise reading a plurality of separate ID from the VU 105 and/or DT 106 thereof. For example, interrogating a VU 105 may comprise reading a first ID 131 (e.g., an NDC) and a separate second ID 132 (e.g., a lot ID, such as a lot number, batch number or the like). Alternatively, or in addition, interrogating a VU 105 may comprise extracting a plurality of ID from information read from the DT 106. For example, interrogating a VU 105 may comprise a) acquiring identification data from a DT 106 of the VU 105, such as a GTIN, UPC, or the like, and b) extracting a plurality of ID from the acquired identification data. The extracting may comprise 1) extracting a first ID 131 determinative of beneficial product type from the acquired identification data, such as an NDC, RxNorm, or the like, and 2) extracting a second ID 132 determinative of the lot or batch of the beneficial product from the acquired identification data.

The output module 144 may comprise any suitable means for implementing dispense or fulfilment operations. As used herein, fulfilment operations comprise and/or refer to operations to dispense VU 105 to patients 102. A fulfilment operation may, for example, comprise fulfilling a prescription for the patient 102 by, inter alia, dispensing beneficial products prescribed for the patient as or within a suitable VU 105. A fulfilment operation may comprise outputting provisioning or fulfillment data. The fulfilment data may be encoded on a medium associated with the VU 105, such as a DT 106, as disclosed herein. For example, a fulfilment operation may comprise printing information pertaining to a patient prescription on a label (e.g., a text DT 106); the label may be affixed to the VU 105 when the VU 105 is dispensed to the patient 102. The output module 144 may comprise any means for outputting provisioning data. The output module 144 may, for example, comprise and/or be coupled to one or more output devices, which may include, but are not limited to: a printer, an ink printer, a thermal printer, a label printer (e.g., a device configured to print on a label and/or DT 106 of a VU 105), a device configured to write data to VU 105 (e.g., a device configured to store electronic data on and/or within a DT 106), and/or the like. For example, the output module 144 may comprise a print device configured to print data on a printable area of a VU 105. Alternatively, or in addition, the output module 144 may comprise a device configured to produce DT 106 configured to be applied to VU 105 (e.g., print labels that can be attached to the VU 105, such as adhesive labels or the like).

In some implementations, the output module 144 may be further configured to generate dispense tokens 134 corresponding to fulfillment and/or provisioning operations. A dispense token 134 may be configured to identify a VU 105 to be dispensed to a patient 102 in a provisioning operation, as disclosed in further detail herein. A dispense token 134 may comprise any information pertaining to a provisioning operation. For example, a dispense token 134 may identify the type of beneficial product to be dispensed (per the first ID 131), identify the dispensing apparatus 110 to be used to fulfill the provisioning operation, designate the compartment 120 from which the VU 105 comprising the product should be retrieved (per the second ID 132), identify the patient 102 to which the VU 105 is to be dispensed (and/or comprise identifying information pertaining to the patient 102), and so on. The dispense token 134 may comprise human-readable information. Alternatively, or in addition, the dispense token 134 may comprise machine-readable data, such as a QR code or the like, as disclosed herein. A dispense token 134 may be generated in any suitable form. For example, a dispense token 134 may comprise a physical dispense token (DT) 135. A physical DT 135 may comprise a dispense token 134 embodied on a physical medium, such as paper, a receipt, a card, a slip, or the like. A physical DT 135 may be generated by a suitable output device (e.g., the output module 144), such as a printer, or the like. Alternatively, or in addition, a dispense token 134 may comprise a virtual DT 136. A virtual DT 136 may comprise electronic data configured for display and/or access through an application 137. The application 137 may be configured to interface with the dispensing apparatus 110 and/or BPM 160 through the network 109. The operator computing device 138 may comprise any suitable computing means, including, but not limited to: a portable computing device, a smart phone, a PDA, a portable terminal, a tablet computer, a notebook computer, and/or the like.

As disclosed in further detail herein, a provisioning operation to dispense a specified type of product to a patient 102 may comprise: a) receiving a request to provision the specified type of product to the patent 102 at the BPM 160, e.g., through the network 109, b) selecting a compartment 120 from which to dispense a VU 105 comprising the specified type of product, as disclosed in further detail herein, c) generating a dispense token 134 identifying the VU 105 and selected compartment 120, d) reading the dispense token 134, e.g., in response to the operator 103 presenting the dispense token 134 at the DAQ module 142, e) providing access to the compartment 120 designated by the dispense token 134 while blocking access to other compartments 120 of the dispensing apparatus 110, f) verifying the first ID 131 and second ID 132 of a VU 105 retrieved from the designated compartment 120, e.g., in response to the operator 103 presenting the VU 105 at the DAQ module 142, and g) printing a label to be applied to the VU 105 for the patent 102 in response to verifying the first ID 131 and second ID 132, e.g., by use of the output module 144.

The control module 140 may be configured to secure access to compartments 120 of the dispensing apparatus 110, e.g., block access to selected compartments 120. FIG. 1C illustrates an example of a compartment 120 of the dispensing apparatus 110. As shown in FIG. 1C, the compartment 120 may comprise an interior volume 121 configured to hold one or more VU 105. In some implementations, the interior volume 121 may be substantially open, which may enable the compartment 120 to hold VU 105 having a wide range of different shapes and/or sizes. Alternatively, or in addition, the interior volume 121 of a compartment 120 may comprise internal elements 128 configured to receive, hold, and/or secure particular types of VU 105 within the compartment 120. FIG. 1C illustrates an example of an internal element 128 comprising receptacles 129 configured to hold cylindrically shaped VU 105, such as bottles, pill bottles, or the like. The disclosure is not limited in this regard, however, and could be adapted to utilize any suitable internal elements 128 configured to hold any suitable type of VU 105 and/or VU 105 having any suitable physical configuration. For example, in some implementations, the internal element(s) 128 may be removable and/or interchangeable, which may enable compartments 120 of the dispensing apparatus 110 to be adapted to hold different types of VU 105.

Access to the interior volume 121 of the compartment 120 may be controlled by, inter alia, an access device 122. The access device 122 may comprise any suitable means for controlling access to the interior volume 121 including, but not limited to: a door, a panel, a port, a lock, a mechanical lock, an electronic lock, a magnetic lock, and/or the like. The access device 122 may comprise and/or be coupled to a lock mechanism 122-1 having a locked configuration and an unlocked configuration. When the lock mechanism 122-1 is in the unlocked configuration, the interior volume 121 of the compartment 120 may be accessible (e.g., the compartment 120 may be unsecured). Conversely, when the lock mechanism 122-1 is in the locked configuration, the interior volume 121 of the compartment 120 may be inaccessible (e.g., the compartment 120 may be secured). In the FIG. 1C example, the access device 122 is configured to secure the compartment 120 in the closed or locked configuration. FIG. 1D illustrates an example in which the access device 122 is in the open or unlocked configuration, as disclosed herein.

In some implementations, the access device 122 may be configured to control access to the interior volume 121 through an opening 123 in the compartment 120. The compartment 120 and/or access device 122 may comprise a barrier 122-2 configured to selectively block, close, seal, and/or otherwise control access to the interior volume 121 through the opening 123. The barrier 122-2 may include, but is not limited to a door, a hinged door, a sliding door, a panel, a cover, a lid, and/or the like. The compartment 120 may have an open configuration and a closed configuration. When in the open configuration, the interior volume 121 of the compartment 120 may be accessible, e.g., the barrier 122-2 may be configured such that the interior volume 121 of the compartment 120 can be accessed through the opening 123. Conversely, when in the closed configuration, the interior volume 121 of the compartment 120 may be inaccessible, e.g., the barrier 122-2 may be configured to block, close, seal, secure, and/or otherwise prevent access to the interior volume 121 of the compartment 120 through the opening 123.

The access device 122 may be configured to control access to the interior volume 121 by use of the barrier 122-2. As disclosed herein, the access device 122 may have a locked configuration and an unlocked configuration. In the locked configuration, the access device 122 may be configured to lock and/or secure the barrier 122-2 in the closed configuration. In other words, in the locked configuration, the access device 122 may be configured to lock the barrier 122-2 in the closed configuration, e.g., prevent the barrier 122-2 from transitioning from the closed configuration to the open configuration. In the locked configuration, the access device 122 may, therefore, cause the barrier 122-2 to block, close, seal, secure, and/or otherwise prevent access to the interior volume 121 of the compartment 120 through the opening 123. In the unlocked configuration, the access device 122 may be configured to allow the barrier 122-2 to transition from the closed configuration to the open configuration (and vice versa). In other words, in the unlocked configuration, the access device 122 may be configured to provide access to the interior volume 121 of the compartment 120 through the opening 123 (and/or the barrier 122-2).

In some implementations, the compartment 120 and/or access device 122 may comprise and/or be coupled to an actuator 125. The actuator 125 may be configured to transition the compartment 120 between the closed configuration and the open configuration (and/or vice versa). Transitioning the compartment 120 to the closed configuration may comprise causing the barrier 122-2 to transition to the closed configuration as disclosed above, e.g., may comprise closing the barrier 122-2 over the opening 123. Transitioning the compartment to the open configuration may comprise causing the barrier 122-2 to transition to the open configuration as disclosed above, e.g., may comprise moving the barrier 122-2 such that the interior volume 121 of the compartment 120 can be accessed through the opening 123.

The actuator 125 may be configured to transition the compartment 120 to the open configuration in response to the access device 122 transitioning to the unlocked configuration (e.g., the actuator 125 may at least partially open the barrier 122-2). Conversely, the actuator 125 may be configured to transition the compartment 120 to the closed configuration in response to the access device 122 transitioning to the locked configuration. For example, the actuator 125 may close the barrier 122-2 so that the lock mechanism 122-1 can be engaged to lock and/or secure the barrier 122-2 over the opening 123. The actuator 125 may comprise any suitable actuation means including, but not limited to: a mechanical actuator, an electrical actuator, a servo, and/or the like.

In some examples, the compartment 120 may further include one or more HMI components (a compartment HMI 124). The compartment HMI 124 may be configured to indicate a status of the compartment 120. For example, the compartment HMI 124 may be configured to indicate whether the compartment 120 is currently accessible (e.g., indicate whether the access device 122 of the compartment 120 is in the locked configuration or the unlocked configuration). Alternatively, or in addition, the compartment HMI 124 may be configured to provide information pertaining to VU 105 stored within the compartment 105 (if any), such as identifying information (e.g., name, identifier, or the like), number of VU 105 currently stored within the compartment 105, expiration date of the VU 105, and/or the like. The compartment HMI 124 may comprise and/or be implemented by one or more HMI resources 154, as disclosed herein. For example, the compartment HMI 124 may comprise an LED or other visual status indicator (e.g., green indicating unlocked and red or “off” indicating locked), a display screen, an audio output device (e.g., a speaker), or the like.

In some implementations, the compartment 120 may further comprise a sensor 127. As used herein, a sensor 127 may refer to any suitable means for acquiring information pertaining to the status and/or contents of the compartment 120, which may include, but is not limited to: an optical sensor, an image sensor, an electromagnetic sensing device, a scale, and/or the like. The sensor 127 may, for example, comprise one or more data acquisition devices, as disclosed herein (e.g., an optical reader, an RFID reader, or the like).

The sensor 127 may be configured to determine a state of the access device 122 (e.g., sense whether the access device 122 is in the open or closed configuration, in the locked or unlocked configuration, and/or the like). The sensor 127 may be further configured to acquire information pertaining to contents of the compartment 120. For example, the sensor 127 may be configured to determine a mass or weight of VU 105 disposed within the compartment 120. Alternatively, or in addition, the sensor 127 may comprise and/or be coupled to a computer vision system configured to detect VU 105 within the interior volume 121 of the compartment 120. In some implementations, the sensor 127 may be configured to acquire information from VU 105 disposed within the compartment 120, e.g., the sensor 127 may be configured to read DT 106 of VU 105 within the compartment 120.

Components of the compartment 120 may be operatively and/or communicatively coupled to the control module 140. In some implementations, the compartment 120 may be coupled to the control module 140 by an interface (a compartment interface 126). The compartment interface 126 may be configured to communicatively and/or operatively couple components of the compartment 120 to the data interface 155 of the control module 140. The compartment interface 126 may comprise any suitable communication and/or interface means including, but not limited to: an electrical communication interface, a direct interface, a wired communication interface (e.g., wires coupled to respective components), a wireless interface, a modular communication interface, a serial port, parallel port, a network interface, and/or the like.

As disclosed in further detail herein, the compartment interface 126 may be configured to receive commands from the control module 140, which may include, but are not limited to, commands to: close the compartment 120 (e.g., transition to the closed configuration by use of the actuator 125), secure the compartment 120 (e.g., close and/or lock the compartment 120 by use of the access device 122), unlock the compartment 120 (e.g., configure the access device 122 to provide access to the interior volume 121 of the compartment), open the compartment 120 (e.g., unlock and/or open the compartment 120 by use of the actuator 125), and/or the like. The compartment interface 126 may be further configured to communicatively and/or operatively couple the compartment HMI 124 to the control module 140. The compartment interface 126 may be configured to receive commands pertaining to information to be provided through the compartment HMI 124, as disclosed herein.

Although particular examples of dispensing compartments 120 are described herein, the disclosure is not limited in this regard and could be adapted to utilize any suitable type of compartment 120, e.g., may utilize compartments 120 having any suitable shape, dimensions, physical characteristics, and/or the like. For example, FIGS. 1E-1F illustrate examples of drawer-type compartments 120. FIG. 1E illustrates the drawer-type compartment 120 is a closed or locked configuration (e.g., secured by lock mechanism 122-1). When in the closed or locked configuration, access to the interior volume 121 may be secured by an enclosure of the compartment 120, as illustrated in FIG. 1E.

The control module 140 may release the lock mechanism 122-2, which may allow the compartment 120 to transition from the closed configuration to the open configuration illustrated in FIG. 1F, e.g., on rails or other suitable means. In some implementations, the compartment 120 and/or access device 122 may further comprise an actuator 125, which may be configured to transition between the open and closed configuration in response to commands from the control module 140, as disclosed herein. When in the open configuration, the interior volume 121 of the compartment 120 may be accessible through the open opening 123, thereof.

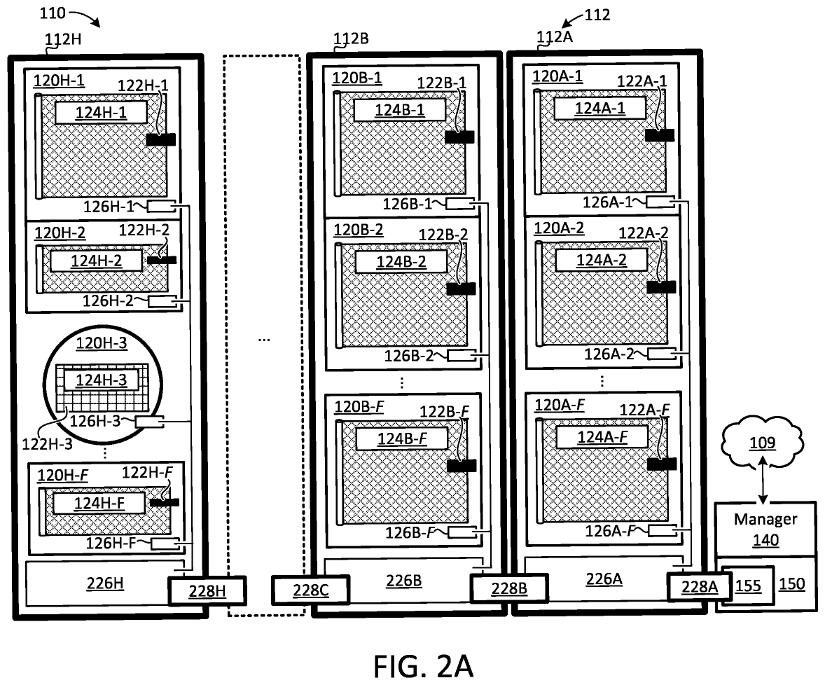

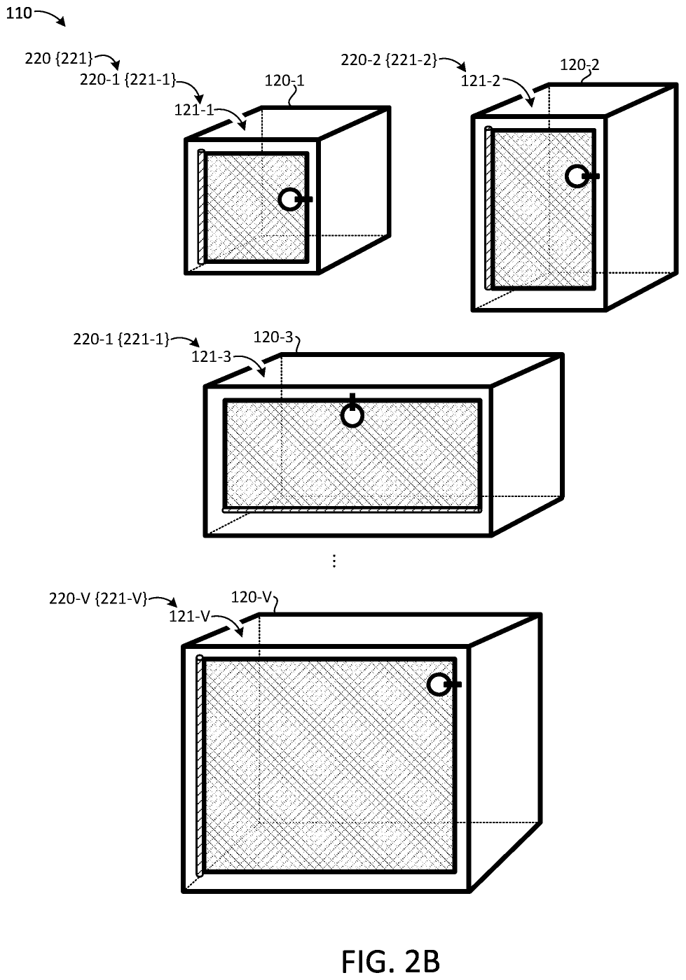

In some implementations, the apparatus 110 may comprise compartments 120 disposed within respective modular structures. FIG. 2 is a schematic block diagram illustrating an example of a modular dispensing apparatus 110. The dispensing apparatus 110 illustrated in FIG. 2 comprises a plurality of modular dispensing components (MDC) 112. In some implementations, each MDC 112 may further comprise an MDC interface 226 configured to operatively and/or communicatively couple compartments 120 of the MDC 112 (and/or components thereof) to the control module 140 and/or other MDC 112 of the apparatus 110.

In the FIG. 2 example, the dispensing apparatus 110 comprises H MDC 112, each comprising F compartments 120; MDC 112A comprises compartments 120A-1 through 120A-F coupled to MDC interface 226A, MDC 112B comprises compartments 120B-1 through 120B-F coupled to MDC interface 226B, MDC 112H comprising compartments 120H-1 through 120H-F coupled to MDC interface 226H, and so on. The disclosure is not limited in this regard, however, and could be adapted for MDC 112 comprising compartments 120 of any type, size and/or configuration. For example, MDC 112H comprises differently sized compartments 120 (e.g., compartments 120H-2 and 120H-F are smaller than compartment 120H-1), compartments 120 having different shapes (e.g., a circular or cylindrical compartment 120H-3), and so on.

A dispensing apparatus 110 may comprise compartments 120 having a plurality of different internal configurations 220. As used herein, the internal configuration 220 of a compartment 120 may refer to characteristics of the internal volume 121 of the compartment 120, which may include, but are not limited to, the dimensions of the internal volume 121 (e.g., x, y, and z measurements, height, width, and depth, measurements, or the like), a shape or geometry of the internal volume 121 (e.g., cubic, cylindrical, triangular, or the like), internal element(s) 128 (if any), within the internal volume 121, and so on. The internal configuration 220 of a compartment 120 may, therefore, determine a size 221 of the compartment 120, where the size 221 of the compartment 120 refers to the internal volume or storage capacity of the compartment 120.

FIG. 2B illustrates examples of compartments 120 of a dispensing apparatus 110 (and/or MDC 120), as disclosed herein. Compartments 120-1 through 120-V of FIG. 2B may have different internal configurations 220 (e.g., internal configurations 220-1 through 220-V, respectively). As disclosed herein, the internal configuration 220 of a compartment 120 may correspond to the size, shape, and/or internal configuration of the internal volume 121 thereof. The internal configuration 220 of a compartment 120 may, therefore, indicate the size and/or storage capacity of the compartments 120.

As illustrated in FIG. 2B, a dispensing apparatus 110 (and/or MDC 120) may comprise V different types of compartments 120 having V different internal configurations 220. The dispensing apparatus 110 may comprise one or more compartments 120-1 with internal volumes 121-1 having internal configuration 220-1, compartments 120-2 with internal volumes 121-2 having internal configuration 220-2, compartments 120-3 with internal volumes 121-3 having internal configuration 220-3, compartments 120-V with internal volumes 121-V having internal configuration 220-V, and so on. The dispensing apparatus 110 may, therefore, comprise compartment 120 of V different sizes, from a smallest size (e.g., compartments 120-1 having internal configuration 220-1) to a largest size (e.g., compartments 120-V having internal configuration 220-V). Although FIG. 2B illustrates compartments 120 having rectangular, or cubic internal configurations 220, the disclosure is not limited in this regard, and could include compartments 120 having internal volumes 121 having any suitable shape and/or internal element(s) 128, e.g., cylindrical compartments 120, conical compartments 120, polygonal compartments 120, or the like.

Referring back to FIG. 2A, the dispensing apparatus 110 may further comprise one or more couplers 228. As used herein, a coupler 228 may comprise and/or refer to any suitable means for physically, operatively, and/or communicatively coupling MDC 112 (and/or compartments 120 thereof). A coupler 228 may include, but is not limited to: physical coupling means (e.g., physical or structural coupling elements, bolts, screws, plates, latches, clips, and/or the like), electro-mechanical coupling means (e.g., a magnetic coupling), communication coupling means, an electrical communication interface, a direct interface, a wired communication interface, a wireless communication interface, a modular communication interface, a serial port, parallel port, a network interface, and/or the like. The couplers 228 may be configured to operatively and/or communicatively couple MDC 112A-H to the control module 140, such that the control module 140 can individually control respective compartments 120A-1 through 120H-F. In the FIG. 2 example, the dispensing apparatus 110 comprises H couplers 228 configured to physically, operatively, and/or communicatively couple MDC 112A-H in a serial or daisy-chain configuration; coupler 228A is configured to couple the MDC 112A to the control module 140 (and/or data interface 155 thereof), coupler 228B is configured to couple MDC 112B to MDC 112A (and the control module 140), coupler 228C is configured to couple MDC 112B to a next MDC 112 (if any), coupler 228H is configured to couple MDC 112H to the control module 140 (through, inter alia, MDC 112A and 112B), and so on. The disclosure is not limited in this regard, however, and could be adapted to physically, operatively, and/or communicatively couple MDC 112 (and/or compartments 120 thereof) in any suitable manner and/or configuration.

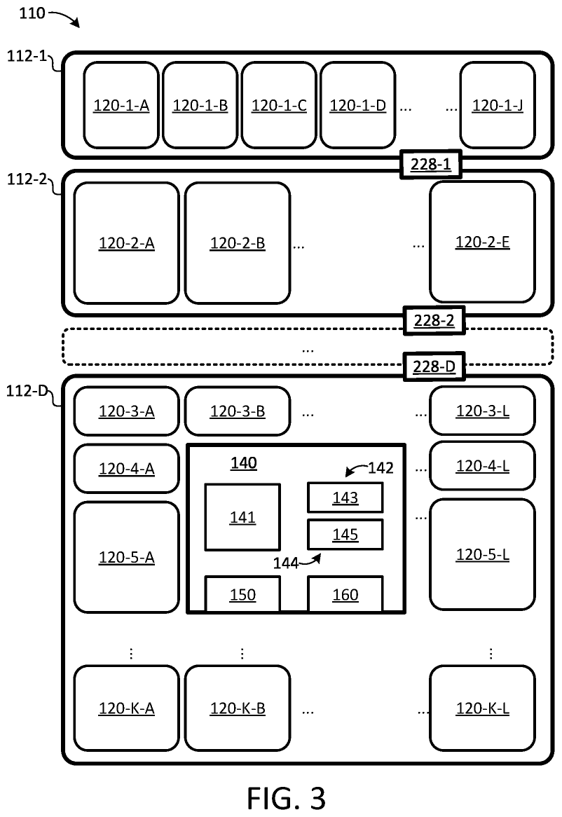

For example, in some implementations, a dispensing apparatus 110 may comprise MDC 112 arranged in other configurations, such as a vertical orientation as illustrated in FIG. 3. In the FIG. 3 example, the dispensing apparatus 110 comprises MDC 112-1 through 112-D; MDC 112-1 comprises relatively small compartments 120-1-A through 120-1-1 having substantially square cross sections, MDC 112-2 comprises compartments relatively larger compartments 120-2-A through 120-2-E, MDC 112-D comprises compartments 120-3-A through 120-K-L having substantially rectangular cross sections, and so on. The MDC 112-1 through 112-D may be operatively and/or communicatively coupled to the control module 140 of the dispensing apparatus 110 by couplers 228-1 through 228-D, as disclosed herein.

In some implementations, the control module 140 (and/or computing resources 150 thereof) may be disposed within an MDC 112 of the dispensing apparatus 110. In the FIG. 3 example, the control module 140 is implemented within MDC 112-D. The control module 140 may comprise a control HMI 141. The control HMI 141 may be configured to facilitate human interaction with the dispensing apparatus 110. More specifically, the control HMI 141 may be configured to enable authorized personnel 103 to perform a) intake operations to load VU 105 into compartments 120 of the apparatus 110, b) fulfillment operations to dispense VU 105 to patients 102, and so on. As disclosed herein, the control HMI 141 may comprise any suitable HMI components and/or HMI resources 154. The control HMI 141 may, for example, comprise a display screen, touch screen, keyboard, pointing device, and/or the like.

The DAQ module 142 may be configured to interrogate VU 105. As disclosed herein, interrogating a VU 105 may comprise reading and/or validating ID of the VU 105 from a DT 106 (or other data source). More specifically, interrogating a VU 105 may comprise acquiring a plurality of ID, such as a first ID 131 and a second ID 132 of a hierarchical ID scheme 130. The DAQ module 142 may implement interrogation operations by use of one or more acquisition devices. In the FIG. 3 example, the DAQ module 142 comprises and/or is coupled to an optical scanner 143 acquisition device. The optical scanner 143 may be configured to retrieve data optically encoded on DT 106, such as barcodes or the like.

The output module 144 may be configured to implement fulfilment operations. As disclosed herein, a fulfilment operation may comprise encoding data on a VU 105 in preparation for dispensing the VU 105 to a patient 102. The provisioning data may be encoded on a DT 106 of the VU 105, such as a label or the like. In the FIG. 3 example, the output module 144 may be configured to implement fulfilment operations by use of a print device 145. The print device 145 may be configured to print human and/or machine-readable data on respective VU 105 (and/or DT 106 of the respective VU 105). By way of non-limiting example, the print device 145 may comprise a label printer or the like, as disclosed herein.

Referring to FIG. 1A, the control module 140 may comprise and/or be communicatively coupled to a beneficial product manager (BPM) 160 (or manager 160). The BPM 160 may comprise, implement, and/or be embodied by inventory control logic 161. As disclosed in further detail herein, the BPM 160 may be configured to implement intake operations to load VU 105 into apparatus 110, provisioning operations to dispense VU 105 from the apparatus 110, and so on. The BPM 160 may be configured to maintain information pertaining to VU 105 held within, and dispensed from, compartments 120 of the apparatus 110. The BPM 160 may utilize the data store 170 to group VU 105 within compartments 120 of the apparatus 110 in accordance with a hierarchical ID scheme 130. More specifically, the BPM 160 may utilize the data store 170 to ensure that VU 105 are maintained within compartments 120 based on a combination of a plurality of different ID (e.g., a composite ID), such as a combination of at least a portion of the first ID 131 and at least a portion of the second ID 132.

As disclosed above, the first ID 131 may be configured to identify the type of beneficial product contained within the VU 105. In other words, the first ID 131 may be determinative of VU type (or beneficial product type). The first ID 131 may be configured to identify the quantity, concentration, dosage, and/or other characteristics of the beneficial product of the VU 105. The first ID 131 may, for example, comprise an NDC, RxNorm, or other suitable ID. The second ID 132 may be configured to identify the lot or batch of the beneficial product. The combination of the first ID 131 and the second ID 132 of a VU 105 may, therefore, be determinative of a) the type of beneficial product contained within the VU 105 and b) the manufacturing lot or batch of the beneficial product.

The BPM 160 may manage the intake of VU 105 into the apparatus 110. The inventory control logic 161 may be configured to implement intake operations to load VU 105 into the dispensing apparatus 110. The intake operations may comprise a) determining a plurality of identifiers for respective VU 105, comprising a first ID 131 and second ID 132, and b) assigning VU 105 to compartments 120 of the dispensing apparatus 110 such that the VU 105 are grouped within the compartments 102 by both the first ID 131 and the second ID 132. The BPM 160 may implement an assignment algorithm (and/or comprise assignment logic) configured such that compartments 120 of the dispensing apparatus 110 comprise corresponding VU 105. As used herein, “corresponding” VU 105 may comprise and/or refer to VU 105 having identifiers that match and/or correspond with one another per the hierarchical ID scheme 130 disclosed herein. Corresponding VU 105 may, therefore, refer to VU 105 that the same and/or corresponding first ID 131 and second ID 132 (and/or the same and/or corresponding composite ID. In other words, corresponding VU 105 may refer and/or comprise VU 105 comprising beneficial products of the same product type (e.g., same NDC) manufactured in the same batch or lot (e.g., same batch or lot identifier).

Managing VU 105 in accordance with the disclosed hierarchical ID scheme 130 can result in significant technical advantages and address shortcomings of many conventional systems. By way of first non-limiting example, the disclosed hierarchical ID scheme 130 can enable the system 100 to efficiently, and accurately, handle recalls. Often, a recall impacts a particular lot or batch of a beneficial product, e.g., the recall may be due to defect(s) introduced during manufacture of the medication, such as contamination or the like. In these situations, it can be critically important to remove the recalled medication from the apparatus 110 so that it cannot be inadvertently dispensed to patients 102. In systems that maintain VU 105 by NDC alone, this can be a time-consuming, error-prone task, particularly since VU 105 of the same type often have the same, or highly similar, appearance, regardless of lot ID. As such, removing recalled medication may require personnel 103 to inspect all of the medication of a particular type, giving rise to the potential for human error. The systems and methods disclosed herein can address these and other shortcomings. As disclosed above, the BPM 160 may be configured to assign VU 105 to compartments 120 based on a combination of the first ID 131 and the second ID 132; VU 105 may be grouped within compartments 120 of the apparatus 110 by type (e.g., NDC) and lot or batch. Therefore, VU 105 impacted by a recall may be grouped within identified compartment(s) 120 of the apparatus 110 and the recalled VU 105 may be removed by simply emptying the identified compartment(s) 120, without the need for manually inspecting each VU 105 (and without the corresponding potential for human error).

The hierarchical ID scheme 130 can improve other technical aspects of automated inventory management. For example, lot ID often correspond to VU 105 expiration date, e.g., the expiration date of the corresponding beneficial product(s). Since the BPM 160 maintains VU 105 within compartments 120 by a combination of the first ID 131 and the second ID 132, the BPM 160 can enable expired VU 105 to be efficiently and accurately identified and removed from the dispensing apparatus 110. Similarly, the BPM 160 may configure fulfilment operations in accordance with lot-specific characteristics, such as expiration. For example, the BPM 160 may be configured to dispense medication of a particular type per a first-expire-first-out (FEFO) scheme wherein VU 105 closer to their expiration date may be dispensed before VU 105 further from their expiration date. The disclosed FEFO scheme may be more efficient than other schemes based on intake date, such as first-in-first-out (FIFO), since intake date may not correspond to expiration date. For example, a particular VU 105 with an earlier expiration date may be loaded after one or more VU 105 with later expiration dates; under a FIFO scheme, the VU 105 with the later expiration dates may be dispensed first, which may result in the particular VU 105 expiring before being dispensed.

The disclosed hierarchical ID scheme 130 may also enable the system 100 to accurately track the medication dispensed to patients 102 at a higher level of detail. As illustrated above, the system 100 may be configured to track medication dispensed to patients 102 by both medication type and lot, which may enable the system 100 to notify patients 102 of lot-specific events, such as recalls or the like. VU 105 of the same type (but different lot), however, often have a similar, or even identical, appearance, giving rise to the potential for human error. For example, an operator may dispense VU 105 of the correct type (e.g., NDC) but wrong lot or batch to patients 102. Dispensing VU 105 from compartments 120 comprising VU 105 organized by a combination of first ID 131 (e.g., NDC) and second ID 132 (e.g., lot ID) can eliminate the potential for such error.

As illustrated in FIG. 1A, the BPM 160 may maintain information pertaining to VU 105 within a data store 170. The data store 170 may comprise any suitable data storage means, including, but not limited to: NT computer-readable storage, a file system, a network-accessible storage service, a network attached storage (NAS) system, a distributed storage and processing system, a cloud-based storage system, a distributed file system, a virtualized data management system, a database, a database management system (DMBS), a relational DMBS, an XML DMBS, an Object-Oriented DMBS, a streaming database system, a directory system, a Lightweight Directory Access Protocol (LDAP) directory system, and/or the like.

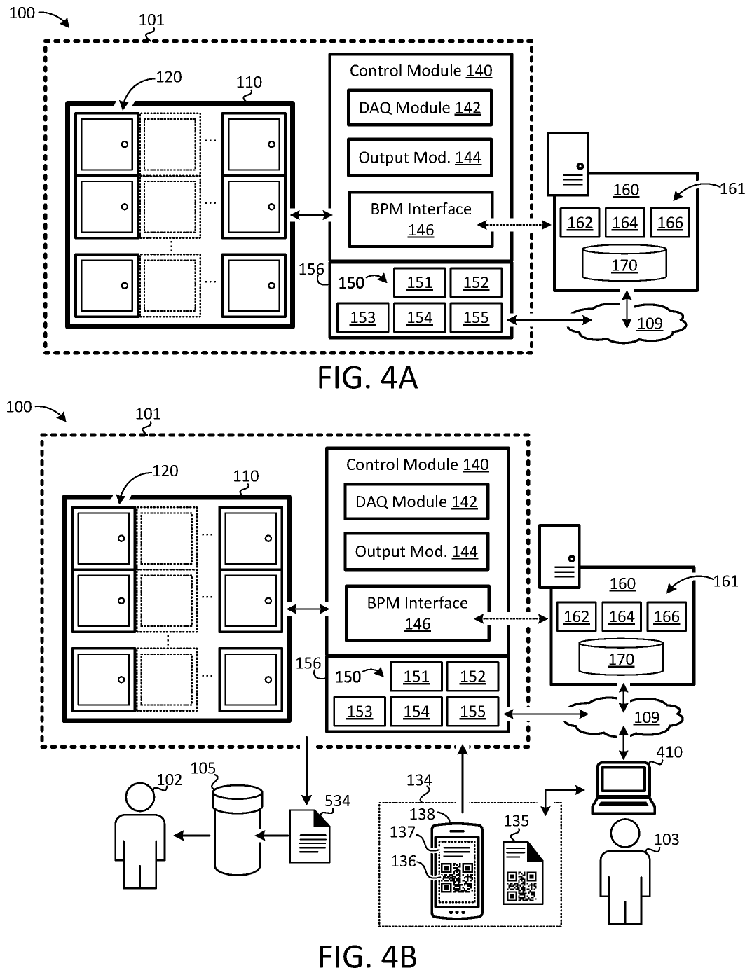

In some implementations, the BPM 160 may be implemented and/or embodied by computing resources 150 of the control module 140. For example, aspects of the BPM 160 and/or data store 170 may be implemented by use of computing resources 150 of the control module 140. Alternatively, or in addition, aspects of the BPM 160 and/or data store 170 may be implemented by one or more external devices and/or systems. For example, the BPM 160 and/or data store 170 may be implemented as a cloud-based, network-accessible service, as illustrated in FIG. 4. In these implementations, the control module 140 may access functionality of the BPM 160 through and/or by use of a BPM interface 146.

As illustrated in FIGS. 1A and 4A-B, the BPM 160 may comprise and/or be coupled to intake logic 162, fulfilment logic 164, and tracking logic 166. The intake logic 162 may be configured to manage intake operations to load VU 105 into the apparatus 110, the fulfilment logic 164 may be configured to manage provisioning operations to dispense VU 105 from the apparatus 110, and the tracking logic 166 may be configured to maintain information pertaining to VU 105 dispensed to respective patients 102.

FIG. 4B is a schematic block diagram illustrating an example of a system 100 configured to fulfill provisioning operations. In the FIG. 4B example, VU 105 may be loaded into compartments 120 of the dispensing apparatus 110 in accordance with the hierarchical ID scheme 130 disclosed herein. In other words, the BPM 160 may be configured to assign VU 105 to compartments 120 such that the VU 105 are grouped within the compartments by composite ID, e.g., by both first ID 131 and second ID 132.

The BPM 160 may be further configured to manage provisioning operations to dispense VU 105 to patients 102. In some implementations, fulfilling a provisioning operation to dispense a specified type of product to a patent 102 may comprise: a) receiving a request to provision the specified type of product to the patent 102 at the BPM 160 and/or BPM interface 146, e.g., through the network 109, b) selecting a compartment 120 from which to dispense a VU 105 comprising the specified type of product, c) generating a dispense token 134 identifying the VU 105, selected compartment 120 and so on, e.g. the patent 102 to which the VU 105 is to be dispensed, d) reading the dispense token 134 at the dispensing apparatus 110, e.g., in response to the operator 103 presenting the dispense token 134 at the DAQ module 142 of the dispensing apparatus 110, e) providing access to the compartment 120 designated by the dispense token 134 while blocking access to other compartments 120 of the dispensing apparatus 110, f) verifying the first ID 131 and second ID 132 of a VU 105 retrieved from the designated compartment 120, e.g., in response to the operator 103 presenting the VU 105 at the DAQ module 142 of the dispensing apparatus 110, g) printing a VU dispense label 534 to be applied to the VU 105 for the patent 102 in response to verifying the first ID 131 and second ID 132, e.g., by use of the output module 144, and h) prompting the operator 103 to apply the VU dispense label 534 to the VU 105 and provide the VU 105 to the patient 102. In some implementations, the operator 103 may receive the dispense token 134 at the dispensing apparatus 110. For example, the BPM 160 may configure the output module 144 to generate a physical DT 135 in response to receiving the provisioning request (and selecting the compartment 120 from which to dispense the specified type of beneficial product). In another example, the BPM 160 may generate a virtual DT 136 for the operator 103, as disclosed herein. The BPM 160 may push the virtual DT 136 to an application 137 deployed on an operator computing device 138, as disclosed herein (and/or the application 137 may be configured to periodically poll the BPM 160 for provisioning requests and/or virtual DT 136).

Alternatively, or in addition, the operator 103 may acquire the dispense token 134 at an operator terminal 410. The operator terminal 410 may be communicatively coupled to the BPM 160 through an electronic communication network, such as the network 109. An operator 103, such as a physician or nurse, may initiate provisioning at the operator terminal 410, e.g., may issue a prescription to the patient 102 at the terminal 410. In response, the terminal 410 may issue a request to the BPM 160, which may identify a dispensing station 110 and/or compartment 120 for use in fulfilling the prescription. The terminal 410 may be further configured to generate a dispense token 134 identifying the selected dispensing station 110 and/or compartment 120, as disclosed herein. The operator 103 may present the dispensing token 134 at the DAQ module 142 of the dispensing station 110, which may provide access to the selected compartment 120 for fulfillment of the provisioning request, as disclosed herein.

The provisioning operations disclosed herein may provide multiple protections against error. The selection of a compartment 120 by the inventory management logic 161 may prevent a human operator 103 from selecting the wrong compartment 120, e.g., selecting a compartment 120 comprising some other type of beneficial product. Similarly, providing access to the selected compartment 120 while blocking access to other compartments 120 may prevent the operator 103 from accessing the incorrect compartment 120. In addition, the use of a computer-readable dispense token 134 may remove the possibility of user transcription error. Scanning the VU 105 retrieved from the selected compartment 120 to validate the first ID 131 and second ID 132 before the VU 105 is provisioned to the patent 102 may further protect against inventory intake error, e.g., obviate the possibility of the incorrect type of VU 105 being loaded into the selected compartment 120 and subsequently retrieved.

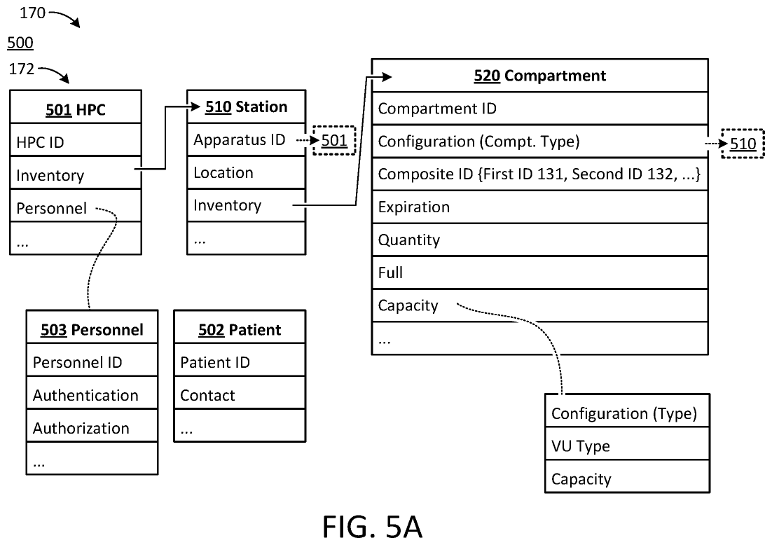

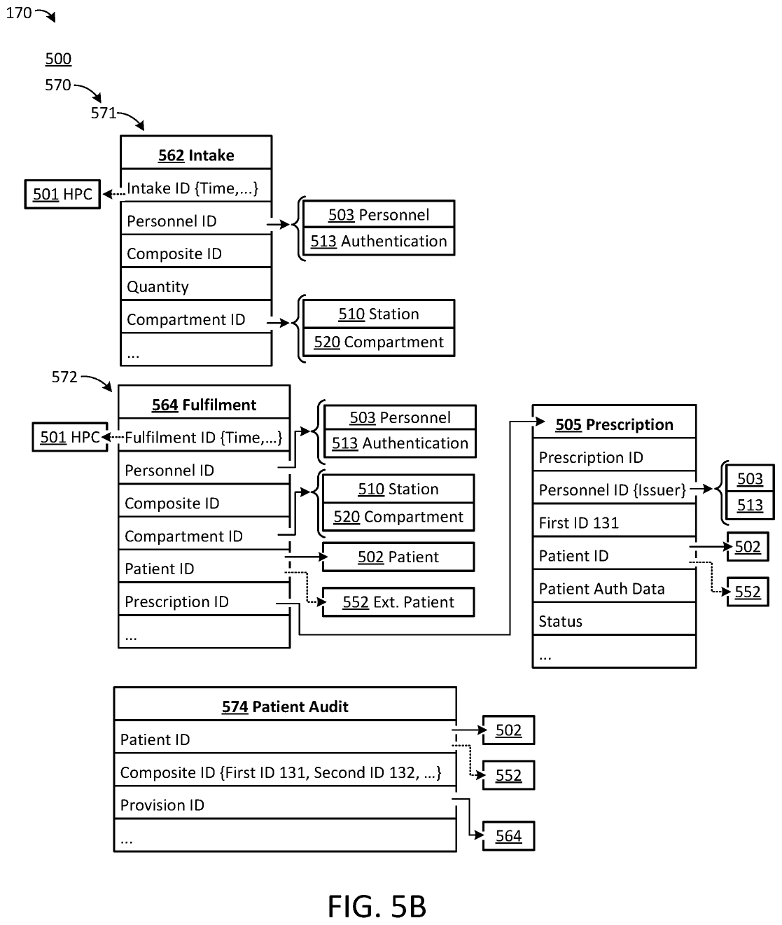

As disclosed herein, the BPM 160 may be configured to store information pertaining to intake, provisioning, and/or tracking operations within the data store 170. FIG. 5A illustrates an example of a schema 500 for maintaining information pertaining to the intake, maintenance, distribution, and/or tracking of VU 105. The data store 170 may comprise non-transitory (NT) records 172 stored on and/or within non-transitory storage media. The NT records 172 may comprise any suitable type of machine-readable information in any suitable format or encoding. The schema 500 may comprise any suitable NT records 172 having any suitable relationships. In the FIG. 5A example, and as disclosed in further detail herein, the NT records 172 maintained in the data store 170 may include, but are not limited to: HCP records 501, patient records 502, personnel records 503, station records 510, compartment records 520, and so on.

The HCP records 501 may comprise information pertaining to respective HCP 101, which may include, but is not limited to: an HCP identifier (e.g., a name, address, contact information, and/or other information of the HCP 101), information pertaining to authorized personnel 103 of the HCP 101, information pertaining to an inventory of beneficial products managed by the HCP 101 (e.g., information pertaining to an inventory of VU 105 managed by the HCP 101), and so on.

In some implementations, the data store 170 may be further configured to maintain information pertaining to patients of the HCP 101. The BPM 160 may be configured to record information pertaining to patients 102 in respective patient records 502. A patient record 502 may comprise any suitable information pertaining to a patient, including a patient ID (e.g., a patient name, address, and/or the like), patient contact information, and so on. Alternatively, or in addition, the BPM 160 may reference patient information maintained in an health care data store (HCDS). The HCDS may be separate and/or independent of the system 100 and/or data store 170. In these implementations, patient records 502 may be omitted from the data store 170, may be configured to reference patient information controlled by the HCDS, and/or the like.

The data store 170 may comprise personnel records 503 configured to represent persons authorized to manage beneficial products of the HCP 101, e.g., represent personnel 103 of the HCP 101. A personnel record 503 may comprise a personnel ID, authentication data, authorization data, and so on. A personnel ID may comprise any suitable information for identifying HCP personnel 103 within the system 100 (and/or data store 170). Personnel ID may comprise and/or be derived from any suitable information pertaining to HCP personnel 103, such as a name, address, contact information, email address, user name, login name, account name, employee ID, human resources (HR) ID, and/or the like.

In some implementations, personnel records 503 may further comprise and/or reference authentication and/or authorization information. The authentication information may comprise information pertaining to mechanisms by which the system 100 may authenticate the identity of the personnel 103, such as a password, a password hash, a shared secret, a biometric, multi-factor authentication data, proximity authentication data (NFC authentication data), and/or the like. The authorization information may specify tasks or operations that personnel 103 are authorized to perform, such as tasks to load VU 105 into specified dispensing apparatus 110, dispense VU 105 to patients 102 from specified apparatus 110, and/or the like.

An HCP record 501 may be associated with zero or more station records 510, each corresponding to a respective dispensing apparatus 110 of the HCP 101. A station record 510 may comprise any suitable information pertaining to a dispensing apparatus 110, including, but not limited to: an identifier of the apparatus 110, a location of the apparatus 110 (e.g., a physical location of the apparatus 110), personnel 103 authorized to operate and/or manage the apparatus 110, an inventory of the apparatus 110, and so on.

The example station record 510 illustrated in FIG. 5A comprises an apparatus ID. The apparatus ID may comprise any suitable information for identifying the apparatus 110 within the system 100 (and/or data store 170), such as a unique identifier, a name, a serial number, and/or the like. The apparatus ID may comprise and/or reference the HCP 101 associated with the dispensing apparatus 110, e.g., may comprise an HCP ID of the HCP record 501.

In the FIG. 5A example, the station record 510 further comprises location information. The location information may comprise any suitable information pertaining to the physical (or virtual) location of the dispensing apparatus 110, including, but not limited to: a physical address of the dispensing apparatus 110, information pertaining to the physical location of the apparatus 110 within a building or other structure, coordinates of the dispensing apparatus 110, a network address of the dispensing apparatus 110 (e.g., an address of a network interface of the dispensing apparatus 110), and/or the like.

The station records 510 may further comprise information pertaining to the inventory of beneficial products (e.g., VU 105) held within respective dispensing apparatus 110. In the FIG. 5A example, information pertaining to the inventory of the dispensing apparatus 110 of the illustrated station record 510 may be maintained within respective compartment records 520 of the data store 170. The compartment records 520 may be configured to represent respective compartments 120 of the dispensing apparatus 110. A compartment record 520 may comprise any suitable information pertaining to a compartment 120 (and/or VU 105 maintained within the compartment 120, if any), which may include, but is not limited to: an identifier of the compartment 120, information pertaining to the VU 105 held within the compartment 120, identifier(s) of the VU 105 held within the compartment 120 (e.g., composite ID, first ID 131, second ID 132, and so on), an expiration date of the VU 105, a quantity of VU 105 held within the compartment 120, an indication of whether the compartment 120 is full (e.g., is fully occupied or has available capacity), a capacity of the compartment 105, and so on.

In some implementations, the data store 170 may be configured to model the configuration of modular dispensing apparatus 110. In these implementations, the compartment records 520 of a modular dispensing apparatus 110 may be organized and/or arranged by MDC 112. For example, the station record 510 may comprise and/or reference module records configured to represent a respective MDC 112 of the dispensing apparatus 110, each comprising compartment records 520 configured to represent respective compartments 120 of the respective MDC 112 (module records not shown in FIG. 5A to avoid obscuring details of the illustrated examples).

The example compartment record 520 illustrated in FIG. 5A comprises a compartment ID. The compartment ID may be configured to identify the compartment 120 within the dispensing apparatus 110 (and/or MDC 112). The compartment ID may, for example, specify a physical location of the compartment 120. In some implementations, the compartment ID may further comprise and/or reference the station record 510 of the dispensing apparatus 110, e.g., may comprise an apparatus ID or the like.

In some implementation, the data store 170 may be further configured to maintain information pertaining to the configuration and/or characteristics of respective compartments 120. In the FIG. 5A example, the compartment record 520 comprises configuration data. The configuration data may comprise any suitable information pertaining to the configuration and/or characteristics of the compartment 120, which may include, but is not limited to: compartment type, the physical configuration of the compartment 120, the size and/or physical configuration of the interior volume 121 of the compartment 120, compartment shape (e.g., rectangular, cylindrical, etc.), the shape of the interior volume 121 of the compartment 120, dimensions of the compartment 120, dimensions of the interior volume 121 of the compartment 120, the physical configuration of the opening of the compartment 120, dimensions of the opening, characteristics of internal element(s) 128 of the compartment 120 (if any), the types of VU 105 that are suitable for storage within the compartment 120, and/or the like.

In some implementations, the BPM 160 may be configured to determine the types of VU 105 that are suitable for storage within respective compartments 120 based, at least in part, on configuration data of the compartments 120. Alternatively, or in addition, the configuration data of the compartment records 520 specify the types of VU 105 that are suitable for storage within respective compartments 120, as disclosed above. In a first non-limiting example, the configuration data may indicate that the interior volume 121 of the compartment 120 comprises internal elements 128 configured to receive and/or hold particular types of VU 105 (e.g., may comprise internal elements 128 configured to hold cylindrically shaped VU 105, such as pill bottles or the like, as illustrated in FIG. 1C). Conversely, in a second non-limiting example, the configuration data may indicate that the interior volume 121 of the compartment 120 is substantially open and, as such, is suitable to receive and/or hold VU 105 having a range of different physical configurations.

The compartment records 520 may be further comprise information pertaining to VU 105 currently being held within respective compartments 120 (if any). The example compartment record 520 illustrated in FIG. 5A comprises a composite ID. The composite ID may comprise and/or be derived from a plurality of ID of the VU 105 in accordance with the hierarchical ID scheme 130 disclosed herein. In other words, the composite ID of a VU 105 may be derived and/or comprise at least a portion of a) the first ID 131 of the VU 105 (e.g., NDC of the VU 105), b) the second ID 132 of the VU 105 (e.g., lot ID of the VU 105), and so on. The composite ID of a VU 105 may, therefore, determine both a) the type of beneficial product within the VU 105 and b) the lot or batch of the beneficial product. Accordingly, a composite ID may comprise and/or be referred to as a hierarchical ID, a combined ID, a multi-level ID, or the like. Alternatively, or in addition, the compartment record 520 may comprise the plurality of ID shared by the VU 105 therein, e.g., may comprise the first ID 131, second ID 132, and so on.

In some implementations, the compartment records 520 may comprise additional information pertaining to the VU 105. For example, the compartment record 520 illustrated in FIG. 5A may indicate the expiration date of the VU 105 being held within the compartment 120. The expiration date may be derived from the composite ID. For example, the expiration date of the VU 105 may be derived from and/or based on the second ID 132 of the VU 105 (e.g., the lot ID of the VU 105), as disclosed herein.

The inventory control logic 121 may be configured to maintain a “full” flag within respective compartment records 520. The full flag of a compartment 120 may indicate whether the compartment 120 is full or has available storage capacity. The full flag may be set to “TRUE” in response to user input indicating that an attempt to load an addition VU 105 failed, e.g., the VU 105 could not be loaded into the compartment 120 and may be set to “FALSE” otherwise, e.g., in response to dispensing a VU 105 from the compartment 120.

Alternatively, or in addition, the compartment records 520 may be further configured to indicate a capacity of respective compartments 120. As used herein, the capacity of a compartment 120 may refer to a quantity, amount, and/or number of VU 105 that the compartment 120 is configured to receive and/or hold within the interior volume 121 thereof. The capacity and quantity fields of a compartment record 520 may, therefore, be compared to determine whether the compartment has available storage capacity (e.g., whether the compartment 120 is full) and, as such, may replace and/or augment the full flag disclosed herein.

The capacity of a compartment 120 may vary by VU type. In some implementations, a compartment 120 may comprise internal elements 128 configured to receive or hold specified types of VU 105, such as internal elements 128 comprising receptacles to hold cylindrical VU 105, as illustrated in FIG. 1C. In these implementations, the capacity of the compartment 120 may correspond to the capacity of the internal elements 128; by way of non-limiting example, a compartment 120 comprising M internal elements 128, each comprising (H×W) receptacles 129 may have a capacity of M×(H×W).

In other non-limiting implementations, the interior volume 121 of the compartment 120 may be substantially free from internal elements 128. The capacity of such compartments 120 to hold a type of VU 105 may be based, at least in part, on characteristics of the particular type of VU 105, such as the size of the VU 105, physical configuration of the VU 105 (e.g., shape of the VU 105), dimensions of the VU 105, volume of the VU 105, and/or the like. For example, an upper end estimate for the capacity of a compartment 120 to hold a particular type of VU 105 may be calculated by dividing the interior volume 121 of the compartment by the volume of the particular type of VU 105. In some implementations, the BPM 160 may learn the capacity of respective types of compartments 120 to hold respective types of VU 105. The BPM 160 may, for example, learn compartment capacity information based on operator feedback (e.g., operator testing, experience, and/or expertise). An operator may, for example, indicate the quantity of particular types of VU 105 that can be held in particular types of compartments 120, indicate the point at which a compartment 120 should be considered to be at full capacity, and so on. Information pertaining to the capacity of respective types of compartments 120 to hold respective types of VU 105 may be maintained within the data store 170, as illustrated in FIG. 5A. Although examples of schema 500 and NT records 172 for managing VU 105 are described herein, the disclosure is not limited in this regard and could be adapted to maintain information pertaining to the intake, management, distribution, and tracking of VU 105 in any suitable data format, schema, and/or technique.

In some implementations, the BPM 160 may be further configured to maintain information pertaining to the management and distribution of beneficial products by the HCP 101. As used herein, information pertaining to the management and distribution of beneficial products may be referred to as audit data 570. FIG. 5B illustrates examples of audit data 570. In the FIG. 5B example, the audit data 570 may be defined in accordance with the schema 500 disclosed herein. Alternatively, aspects of the audit data 570 may be defined in one or more different, separate schema. In some implementations, the BPM 160 may record audit data 570 within the data store 170. Alternatively, or in addition, the BPM 160 may maintain portions of the audit data 570 within separate non-transitory storage resources, such an audit store that is separate and/or independent of the data store 170 (audit store not shown in FIG. 5B to avoid obscuring details of the illustrated examples). For example, in some implementations, the BPM 160 may record portions of the audit data 570 within both the data store 170 and the audit store, e.g., the BPM 160 may mirror or replicate portions of the audit data 570.

The audit data 570 may comprise internal audit data 571 pertaining to the intake, storage and distribution of beneficial products by the HCP 101. As disclosed in further detail herein, the audit data 570 may further comprise patient audit data 572. The patient audit data 572 may be configured to associate patients 102 with VU 105 dispensed to the patients 102. More specifically, the patient audit data 572 may be configured to associate patients 102 with hieratical, composite ID of the VU 105. As used herein, the composite ID of a VU 105 refers to an ID that comprises, includes, incorporates, and/or is derived from a plurality of ID of the VU 105 per the hierarchical ID scheme 130 disclosed herein. For example, the composite ID of a VU 105 may comprise the first ID 131 of the VU 105, the second ID 132 of the VU 105, and so on. The composite ID of a VU 105 may, therefore, identify the type of beneficial product associated with the VU 105 (per the first ID 131) and identity the lot or batch of the beneficial product (per the second ID 132).

FIG. 5B illustrates examples of internal audit data 571, such as intake records 562, fulfillment records 564, and so on. The intake records 562 may be configured to record information pertaining to intake operations, e.g., operations to load VU 105 into a dispensing apparatus 110. An intake record 562 may comprise any suitable information pertaining to an intake operation, including, but not limited to: time and/or date information (e.g., the time and/or date of the intake operation), personnel 103 involved in the intake operation, VU 105 loaded in the intake operation, location of the VU 105 (e.g., compartment 120 and/or dispensing apparatus 110 into which the VU 105 where loaded), and so on.

The intake record 562 illustrated in FIG. 5B may comprise an intake ID. The intake ID may be configured to identify the intake operation within the system 100 (and/or data store 170). The intake ID may comprise and/or be derived from any suitable information, such as a unique identifier, a time or date of the intake operation (e.g., may specify a start and/or end time of the intake operation), the HCP 101 associated with the intake operation, and/or the like. In some implementations, the intake ID may comprise and/or reference the HCP record 501 of the HCP 101 associated with the intake operation.

In the FIG. 5B example, the intake record 562 further comprises personnel ID configured to identify the HCP personnel 103 responsible for performing the intake operation. The intake record 562 (and/or personnel ID thereof) may comprise and/or reference one or more personnel records 503, as disclosed herein. In some implementations, the intake record 562 may further comprise and/or reference personnel authentication records 513. The personnel authentication records 513 may comprise information pertaining to authentication of the personnel 103 responsible for the intake operation, such as the authentication mechanisms used by the personnel 103 (e.g., password, biometric, multi-factor, etc.), authentication time, authentication location, and/or the like.

The intake records 562 may further comprise information pertaining to the beneficial products loaded in the intake operations. The intake record 562 illustrated in FIG. 5B comprises a composite ID for the VU 105 loaded in the intake operation. As disclosed herein, the composite ID may comprise a plurality of ID per the hierarchical ID scheme 130, e.g., may comprise a first ID 131 of the VU 105, a second ID of the VU 105, and so on. In some implementations, the intake records 562 may further specify the quantity of specified VU 105 loaded in the intake operation. Alternatively, intake operations may be audited individually (e.g., the BPM 160 may audit intake of respective VU 105).

The intake records 562 may further identify the compartments 120 into which respective VU 105 were loaded in the intake operations. In the FIG. 5B example, the intake record 562 comprises a compartment ID. The compartment ID may comprise and/or reference the compartment record 520 for the compartment 120 into which the specified VU 105 were loaded in the intake operation. The compartment ID may be further comprise and/or reference information pertaining to the dispensing apparatus 110 of the compartment 120, e.g., may comprise and/or reference a station record 510, as disclosed herein.

The BPM 160 may be configured to record information pertaining to fulfilment operations within the data store 170 (and/or audit data 570). As disclosed herein, a fulfilment operation may comprise and/or refer to an operation to dispense VU 105 to a patient 102. In some implementations, a fulfilment operation may involve fulfilling a prescription for the patient 102. The BPM 160 may be further configured to record information pertaining to prescriptions to be fulfilled using the system 100 within the data store 170 (and/or audit data 570). As illustrated in FIG. 5B, information pertaining to prescriptions may be maintained within prescription records SOS. A prescription record SOS may comprise any suitable information pertaining to a prescription, including, but not limited to information pertaining to personnel 103 responsible for the prescription (the issuer of the prescription), the beneficial products to be dispensed to the patient 102 under the prescription, the patient 102 associated with the prescription, and so on.

The example prescription record SOS illustrated in FIG. 5B comprises a prescription ID, which may be configured to identify the prescription within the system 100 (and/or data store 170), as disclosed herein. The prescription ID may comprise and/or be derived from any suitable information, such as a unique ID, time or date that the prescription was issued, and/or the like. The prescription record SOS may further comprise and/or reference the HCP record 501 of the HCP 101 associated with the prescription.

The example prescription record 505 further comprises a personnel ID. The personnel ID may comprise and/or reference the HCP personnel 103 responsible for issuing the prescription. For example, the personnel ID may reference the personnel record 503 of the doctor or other health-care professional that issued the prescription to the patient 102.

The prescription record 505 may further comprise information pertaining to the patient 102 to which the prescription was issued. The prescription record 505 may comprise a patient ID, which may comprise and/or reference a patient record 502 maintained within the data store 170, as disclosed herein. Alternatively, or in addition, the patient ID may comprise and/or reference an external patient record 552. The external patient record 552 may refer to patient information maintained by an external system, such as a third-party health-care management system (HCMS) or the like.

In the example illustrated in FIG. 5B, the prescription record 505 may identify the type of beneficial product to be dispensed to the patient 102. The prescription record 505 may comprise a first ID 131. As disclosed herein, the first ID 131 may be configured to identify the type, quantity, concentration and/or dosage of beneficial product to be dispensed to the patient 102, e.g., may comprise an NDC, RxNorm, or other suitable identifier.

In some implementations, a prescription record 505 may comprise additional information to, inter alia, facilitate fulfilment of the prescription within the system 100. In the FIG. 5B example, the prescription record 505 may further comprise patient authentication data and/or status information. As disclosed in further detail herein, the patient authentication data (AUTH data) may comprise and/or refer to any information by which the identify of a patient 102 may be determined, such as name, birth date, zip code, age, address, email address, phone number, or the like. Alternatively, or in addition, AUTH data may comprise one or more credentials established by the patient 102, such as a password, personal identification number (PIN), the answer to a “secret question” (e.g., street you grew up on), biometric information, such as a fingerprint, iris scan, voice sample, facial recognition, or the like. In some implementations, the AUTH data may comprise and/or relate to credentialed payment information provided by the patient 102. As used herein, credentialed payment information refers to any information related to a payment transaction, which may include, but is not limited to, credit card information, debit card information, banking information, checking account information, electronic payment information, such as a PAYPAL® credential, or other suitable payment-related information, in such embodiments, the credentialed payment information may be comprised of, or may be used to generate, the patient authentication data. The status information may comprise information pertaining to fulfilment of the prescription by the patient 102, as disclosed in further detail herein.

The BPM 160 may record information pertaining to fulfilment operations to, inter alia, fulfil respective prescriptions within the data store 170 (and/or audit data 570). The BPM 160 may store information pertaining to fulfilment operations within respective fulfilment records 564. A fulfilment record 564 may comprise any suitable information pertaining to a fulfillment operation including, but not limited to: HCP personnel 103 involved in the fulfilment operation (if any), VU 105 dispensed in the fulfilment operation, the dispensing apparatus 110 and/or compartment 120 accessed in the fulfilment operation, the patient 102 to which the VU 105 was dispensed, information pertaining to a prescription associated with the fulfilment operation (if any), and so on.

The example fulfilment record 564 illustrated in FIG. 5B comprises a fulfilment ID, which may be configured to uniquely identify the fulfilment operation within the system 100 (and/or data store 170). The fulfilment ID may comprise and/or be derived from any suitable information, such as a unique identifier, a time or date of the fulfilment operation, and/or the like. The fulfilment ID may further comprise and/or reference the HCP 101 associated with the fulfilment operation, e.g., may comprise and/or reference an HCP record 501, as disclosed herein.

The fulfilment record 564 may further comprise personnel ID, which may comprise and/or reference personnel records 503 of HCP personnel 103 responsible for completing the fulfilment operation (if any). The personnel ID may be further configured to reference authentication records 513 pertaining to authentication of the personnel 103, as disclosed herein.

The fulfilment record 564 may further include information pertaining to the beneficial product(s) dispensed to the patient 102 in the fulfilment operation. In the FIG. 5B example, the fulfilment record 564 comprises a composite ID. The composite ID may comprise a plurality of ID per the hierarchical ID scheme 130 disclosed herein, e.g., may comprise a first ID 131, second ID 132, and so on.

The fulfilment record 564 illustrated in FIG. 5B may further comprise a compartment ID. The compartment ID may comprise and/or reference information pertaining to the compartment 120 accessed during the fulfilment operation. The compartment ID may reference a compartment record 520 and/or station record 510, as disclosed herein.

The fulfilment records 564 may further comprise information pertaining to patients 102 associated with the provisioning operations. The fulfilment record 564 illustrated in FIG. 5B comprises a patient ID. In some implementations, the patient ID may comprise and/or reference a patient record 502 maintained within the data store 170. Alternatively, or in addition, the patient ID may comprise and/or reference an external patient record 552 maintained by an external, third-party patient or HCMS, as disclosed herein.

In some implementations, the fulfilment records 564 recorded by the BPM 160 may pertain to operations to fulfil prescriptions issued to patients 102. In these implementations, the BPM 160 may be further configured to include information pertaining to the prescriptions within the fulfilment records 564. In the FIG. 5B example, the fulfilment record 564 comprises a prescription ID. The prescription ID may comprise and/or reference a prescription record SOS. The prescription record SOS may comprise information pertaining to the prescription associated with the fulfilment operation, as disclosed herein.

As disclosed herein, the BPM 160 may be configured to record patient audit data 572; the patient audit data 572 may be configured to associate patients 102 with VU 105 dispensed to the patients 102. More specifically, the patient audit data 572 may be configured to associate patients 102 with hieratical, composite ID of the VU 105 dispensed to the patients 102, the composite ID comprising first ID 131 determinative of beneficial product type and second ID 132 determinative of beneficial product lot or batch. In some implementations, the BPM 160 may utilize the fulfilment records 564 as patient audit data 572. Alternatively, or in addition, the BPM 160 may record patient audit data 572 within dedicated patient audit records 574.

FIG. 5B illustrates an example of a patient audit record 574. The patient audit record 574 may be configured to associate a patient 102 with a VU 105. More specifically, the patient audit record 574 may be configured to associate the patient 102 with a composite ID of the VU 105 dispensed to the patient 102, wherein the composite ID is determinative of both beneficial product type (per the first ID 131 of the composite ID) and beneficial product lot or batch (per the second ID 132 of the composite ID). The patient 102 of the patient audit record 574 may be identified by a patient ID, which may comprise and/or reference a patient record 502, an external patient record 552, and/or the like, as disclosed herein.

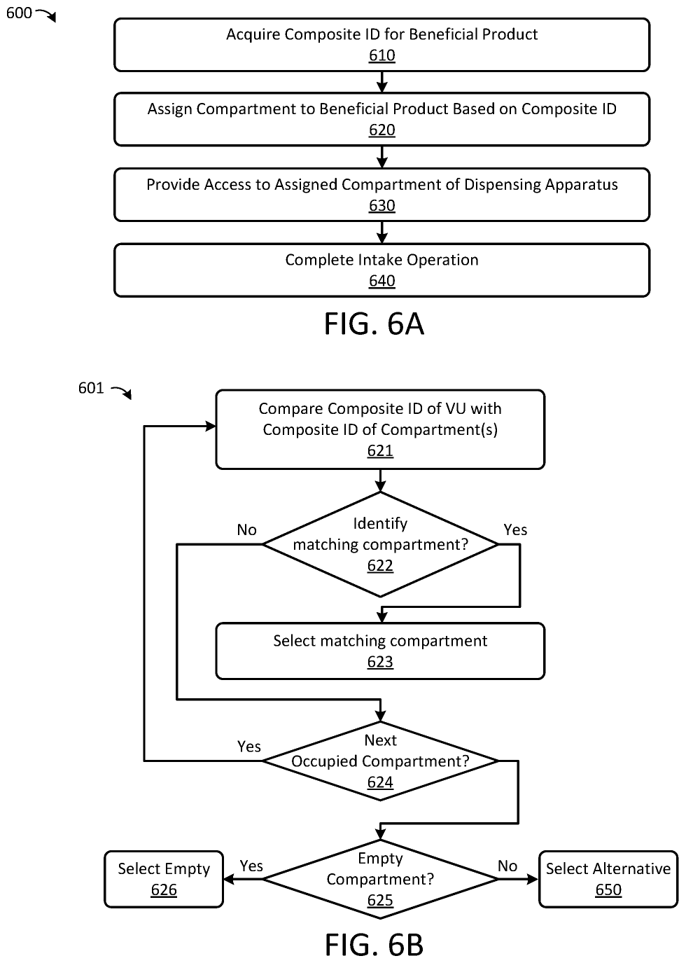

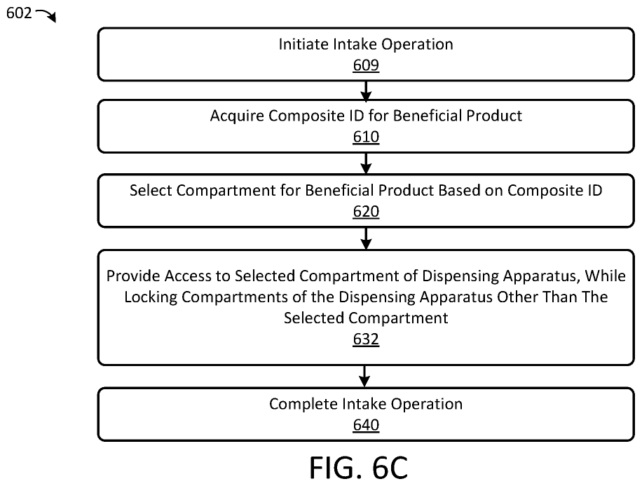

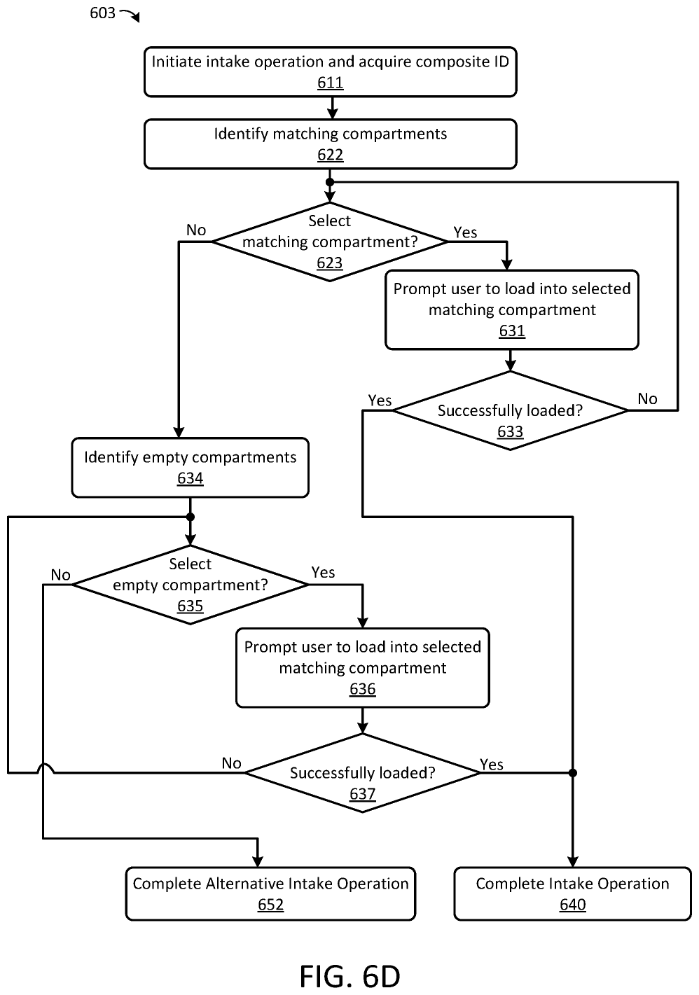

FIG. 6A is a flow diagram illustrating an example of a method 600 for managing intake operations to load VU 105 into a dispensing apparatus 110, as disclosed herein. The steps and/or operations of the method 600 (and/or other methods disclosed herein) may be implemented by a processor of computing device, such as processing resources 151 of an electronic device 156 as illustrated in FIGS. 1A and 4. Steps and/or operations of the disclosed method 600 may be implemented and/or embodied as computer-readable instructions stored on a non-transitory storage medium, such as NT storage resources 153. The computer-readable instructions may be configured to cause a processor to implement the steps and/or operations. Alternatively, or in addition, one or more of the steps and/or operations may be implemented by hardware components, such as logic circuitry, processing circuitry, an ASIC, an FPGA, and/or the like.

At 610, the control module 140 of the dispensing apparatus 110 may be configured to acquire a composite ID for the beneficial product to be loaded into the dispensing apparatus 110. Acquiring the composite ID may comprise initiating the intake operation. The intake operation may be initiated by authorized personnel 103 of the HCP 101. Initiating the intake operation may, for example, comprise authenticating the identity of the personnel 103 through a suitable authentication mechanism, as disclosed herein.

In some implementations, the beneficial product of the intake operation may be contained within a VU 105. In these implementations, acquiring the composite ID of the beneficial product at 610 may comprise interrogating the VU 105. As disclosed herein, interrogating a VU 105 may comprise operations to read, sense, interpret, extract, derive, and/or otherwise acquire data pertaining to the VU 105. The VU 105 may be interrogated at 610 by use of one or more acquisition devices of the DAQ module 142, which may include, but are not limited to an optical scanner, an optical reader, a barcode reader, an OCR device, an OMR device, a computer vision device, an RFID device, an RFID reader, an NFC device, and/or the like. For example, acquiring the composite ID at 610 may comprise placing the VU 105 within scan range of an optical scanner 143 acquisition device or the like.

Acquiring the composite ID at 610 may comprise reading and/or validating ID of the VU 105 from a DT 106 (or other data source). More specifically, at 610 the control module 140 may acquire a plurality of ID, including a first ID 131 and a second ID 132 of the hierarchical ID scheme 130 disclosed herein. The first ID 131 may be configured to identify the type of beneficial product contained within the VU 105. In other words, the first ID 131 may be determinative of VU type (or beneficial product type). The first ID 131 may be configured to identify the quantity, concentration, dosage, and/or other characteristics of the beneficial product of the VU 105, as disclosed herein. The second ID 132 may be configured to identify the lot or batch of the beneficial product contained within the VU 105. The second ID 132 may, for example, comprise a lot ID or other information pertaining to the manufacture of the beneficial product, as disclosed herein.

In some implementations, acquiring the composite ID at 610 may comprise reading a plurality of separate ID from the VU 105 and/or DT 106 thereof. The interrogating at 610 may comprise reading a first ID 131 (e.g., an NDC) and a separate second ID 132 (e.g., a lot ID). Alternatively, or in addition, acquiring the composite ID of the VU 105 at 610 may comprise extracting a plurality of ID from identification information read from the DT 106; at 610, the DAQ module 142 may be configured to a) acquire identification data from a DT 106 of the VU 105, such as a GTIN, UPC, or the like, and b) extract a plurality of ID from the acquired identification data. The extracting may comprise 1) extracting a first ID 131 determinative of beneficial product type from the acquired identification data, such as an NDC, RxNorm, or the like, and 2) extracting a second ID 132 determinative of the lot or batch of the beneficial product from the acquired identification data, such as a lot ID or the like.

At 620, a compartment 120 of the dispensing apparatus 110 may be assigned to receive the beneficial product. The selection of 620 may be determined by intake logic 162 of the BPM 160. The selection of 620 may be configured to group VU 105 within compartments 120 of the dispensing apparatus 110 by composite ID. In other words, the intake logic 162 may be configured to select compartments 120 for VU 105 such that each compartment 120 of the dispensing apparatus 110 comprises VU 105 with matching composite ID (e.g., VU 105 of the same type and lot).

In some implementations, the selection at 620 may be implemented per a predetermined algorithm or set of rules. In a first non-limiting example, the selection at 620 may comprise a) identifying compartment(s) 120 of the dispensing apparatus 110 that match and/or are compatible with the VU 105 (e.g., identifying “matching” or “compatible” compartments 120 that are currently being used to store VU 105 having composite ID that match and/or are compatible with the composite ID of the VU 105 determined at 610), b) attempting to load the VU 105 into one of the identified compartments 120, and c) loading the VU 105 into another, unoccupied compartment 120 of the dispensing apparatus 110 if the VU 105 cannot be loaded into any of the identified compartments 120 (and/or of no matching compartments were identified). As used herein, an “occupied” compartment 120 refers to a compartment 120 that currently holds at least one VU 105 and an “empty” or “unoccupied” compartment 120 refers to a compartment 120 that is not currently holding any VU 105.

In a second non-limiting example, the selection at 620 may comprise a) identifying matching compartment(s) 120, b) selecting a matching compartment 120 for the VU 105, c) attempting to load the VU 105 into the selected matching compartment 120, and d) selecting an unoccupied compartment 120 if the VU 105 cannot be loaded into any of the matching compartments 120. A matching compartment 120 may be selected from a plurality of matching compartments 120 based on any suitable criteria. For example, the matching compartment 120 may be based on one or more of: internal configuration 220 and/or size 221 (e.g., from smallest to largest), quantity of VU 105 currently being held in respective matching compartments 120 (e.g., from lowest occupancy to highest occupancy), available capacity (e.g., select from most available capacity to least available capacity), or the like.

In a third non-limiting example, the selection at 620 may comprise a) identifying matching compartments 120 determined to have available capacity to store the VU 105, b) attempting to load the VU 105 into one of the identified compartments 120, and c) loading the VU 105 into an unoccupied compartment 120 if such attempts fail. The available capacity of a compartment 120 may be based on the internal configuration 220 and/or size 221 of the compartment 120, a quantity of VU 105 currently being held within the compartment 120, internal element(s) of the compartment 120, and so on, as disclosed in further detail herein.

In a fourth non-limiting example, the selection at 620 may comprise a) identifying compatible compartment(s) 120 of the VU 105, b) attempting to load the VU 105 into one of the identified compartments 120, and c) selecting an unoccupied compartment 120 if the VU 105 cannot be loaded into any of the identified compartments 120. In some implementations, the unoccupied compartment 120 of the dispensing apparatus 110 may be selected from a plurality of unoccupied compartments 120. The selection may be based on any suitable criteria, including the internal configuration 220 and/or the size 221 of the unoccupied compartments 120. For example, the intake logic 162 may be configured to select a smallest unoccupied compartment 120 and, if the VU 105 cannot be loaded into the smallest unoccupied compartment 120, select a next larger unoccupied compartment 120, and so on.

In some embodiments, such as the non-limiting examples disclosed above, the inventory control logic 161 may implement hierarchical selection criteria. For example, the inventory control logic 161 may implement a first selection based on a first criterion such as internal configuration 220 or size and a second based on a second criterion. Application of the first criterion may result in selection of a first set of compartments 120, e.g., may result in selection of empty compartments 120 having a same size and/or internal configuration 220. Application of the second criterion may result in selection of a compartment 120 from the first set of compartments 120. The second criterion may select the compartment 120 based on a deterministic criterion, such as a sequential order of the compartments 120, logical addresses of the compartments 120, the MDC 112 of the compartment 120 (the second criterion may be configured to fill compartments 120 within respective MDC 112 before utilizing compartments 120 within other MDC 112), the physical proximity of the compartment 120 to other, occupied compartments 120, and/or the like. Alternatively, or in addition, the second criterion may comprise a non-deterministic criterion, such as random selection, pseudo-random selection, and/or the like.

In some examples, the selection at 620 may be implemented per the method 601 illustrated in FIG. 6B. In the FIG. 6B example, the intake logic 162 may compare the composite ID determined for the VU 105 to composite ID of compartments 120 of the dispensing apparatus 110 at 621 (e.g., to identify compartments 120 that match the composite ID of the VU 105). The comparing may be implemented by, inter alia, comparing the composite ID to composite IDs of occupied compartment records 520 maintained in the data store 170 (and/or searching the data store 170 for a compartment record 520 matching the composite ID). For simplicity, FIG. 6B illustrates a series of comparisons at 621, each comparison involving a respective occupied compartment 120 of the dispensing apparatus 110. The disclosure is not limited in this regard, however, and could be adapted to implement any suitable comparison type (e.g., may evaluate a plurality of occupied compartments 120 in simultaneously).

At 622, the intake logic 162 may evaluate the comparison of 621 to determine whether the compartment 120 matches the composite ID. The evaluation of 622 may further comprise determining whether the matching compartment 120 has available capacity to store the VU 105. The available capacity may be determined by use of the compartment record 520 for the matching compartment 120, as disclosed herein. If at 622, the intake logic 162 determines that the compartment 120 matches the composite ID of the VU 105 and has available capacity, the flow continues to 623 where the compartment 120 is selected for storage of the VU 105. Otherwise, the flow may continue at 624. If multiple compartments 120 matching the composite ID are identified, the inventory control logic 160 may be configured to select between the matching compartments 120 based on second criterion, as disclosed herein.

At 624, the intake logic 162 may determine whether the dispensing apparatus 110 comprises additional occupied compartments 120 for evaluation. If so, the flow may continue back at 621; otherwise, the flow may continue at 625.

At 625, the intake logic 162 may determine whether the dispensing apparatus 110 comprises an empty compartment 120 suitable for storage of the VU 105. If so, the intake logic 162 may select the empty compartment 120 for storage of the VU 105 at 626; otherwise, the flow may continue at 650. If multiple empty compartments 120 are identified at 626, the inventory control logic 160 may be configured to select between the empty compartments 120 based on hierarchical selection criteria, as disclosed herein. By way of non-limiting example, the first criterion may be configured to identify empty compartments 120 by internal configuration 220 and/or size, and the second criterion may select between the identified compartments 120 based on another, different characteristic, such as sequential order, physical proximity to other occupied compartments 120, compartment MDC 112, and so on.

At 650, the intake logic 162 may select an alternative storage location for the VU 105. The intake logic 162 may, for example, indicate that the VU 105 should be stored within another dispensing apparatus 110 or the like. The alternative storage mechanism may comprise attempting to store the VU 105 within a different dispensing apparatus 110, designating the VU 105 for “external storage,” or the like. As used herein, “external storage” refers to storage of a VU 105 in a storage location other than a dispensing apparatus 110, MDC 112, compartment 120, or the like.

Referring back to FIG. 6A, at 630, the control module 140 may provide access to the compartment 120 selected for the VU 105 at 620. At 630, the control module 140 may configure the access device 122 of the selected compartment 120 to transition from the locked configuration to the unlocked configuration (e.g., unlock the compartment 120). In some implementations, the control module 140 may be further configured to open the selected compartment 120 by use of an actuator 125; the control module 140 may configure the actuator 125 to, at least partially, transition the compartment 120 from a closed configuration to an open configuration, as disclosed herein.

Providing access to the selected compartment 120 at 630 may further comprise identifying and/or indicating the selected compartment 120. The control module 140 may be configured to provide a visual, auditory, and/or other suitable indication to identify the selected compartment 120 and/or distinguish the selected compartment 120 from other compartments 120 of the dispensing apparatus 110.

In some implementations, the control module 140 may indicate the selected compartment 120 by use of one or more compartment HMI 124. The control module 140 may, for example, cause the compartment HMI 124 of the selected compartment 120 to generate a notification or queue prompting personnel 103 of the HCP 101 to load the VU 105 into the selected compartment 120. The control module 140 may cause the compartment HMI 124 to produce any suitable prompt, including, but not limited to: a visual indication, such as a visual open indication (e.g., a green light), a visual message (e.g., presenting a message identifying the selected compartment 120 and/or prompting personnel 103 to load the selected compartment 120 on a display screen or other visual HMI device), an audio queue or prompt (e.g., producing an audible tone or message identifying the selected compartment 120 and/or prompting personnel 103 to load the selected compartment 120), and/or the like.

The control module 140 may be further configured to indicate the selected compartment 120 by at least partially opening the selected compartment 120. The control module 140 may, for example, configure the access device 122 and/or actuator 125 of the selected compartment 120 to transition to the open configuration (e.g., at least partially open the barrier 122-2 over the opening 123 of the selected compartment 120).

Providing access to the selected compartment 120 and/or indicating the selected compartment 120 may prevent human error in the intake operation. As disclosed herein, the control module 140 may identify and/or distinguish the selected compartment 120 from other compartments 120 of the dispensing apparatus 110 by, inter alia, unlocking the selected compartment 120, indicating the selected compartment 120 by use of the compartment HMI 124, at least partially opening the selected compartment 120 by use of an actuator 125 thereof, and so on. Accordingly, at 630, the control module 140 may prevent personnel 103 from inadvertently loading VU 105 into compartment(s) 120 other than the selected compartment 120. In this manner, the control module 140 (and BPM 160) may ensure that VU 105 stored within the dispensing apparatus 110 are accurately grouped within respective compartments 120 by both a) beneficial product type (per the first ID 131 of the VU 105), and b) beneficial product lot or batch (per the second ID 132 of the VU 105). Accordingly, the control module 140 (and/or BPM 160) can ensure that, when VU 105 are subsequently dispensed to patients 102 from compartments 120 of the apparatus 110, the patients 102 are receiving VU 105 of known a) beneficial product type (per the first ID 131 of the VU 105 stored within respective compartments 120) and b) beneficial product lot or batch (per the second ID 132 of the VU 105 stored within respective compartments 120).

In some implementations, the control module 140 may be configured to maintain compartments 120 of the dispensing apparatus 110 in a locked configuration until access to the compartments 120 is authorized. In other words, the control module 140 may configure access devices 122 of respective compartments 120 of the dispensing apparatus 110 to remain in the locked configuration until access to the respective compartments 120 is authorized. The control module 140 may authorize access to the selected compartment 120 at 630 (e.g., the control module 140 may provide access to the selected compartment 120 at 630, as disclosed above). Throughout the intake operation of FIG. 6A, access to compartments 120 other than the selected compartment 120 may not be authorized. Therefore, at 630, the control module 140 may configure the compartments 120 other than the selected compartment 120 to remain locked, which may further prevent human error during the intake operation. For example, a human operator may be unable to load the VU 105 into compartment(s) 120 other than the selected compartment 120 since access to the other compartment(s) 120 is locked.

At 640, the intake operation may be completed. Completing the intake operation may comprise determining that the VU 105 has been loaded into the selected compartment 120. Completing the intake operation may comprise receiving, at the control module 140, an indication from personnel 103 that the intake operation has been completed. The completion indication may be received through any suitable means, such as HMI resources 154 of the control module 140, compartment HMI 124 of the selected compartment 120, and/or the like. Alternatively, or in addition, the control module 140 may determine that the intake operation is complete at 640 in response to detecting that the access device 122 of the selected compartment 120 has been returned to the closed (or locked) configuration. In some implementations, completing the intake operation at 640 may comprise verifying that the VU 105 have been placed within the selected compartment 120. The verification may be implemented by use of a sensor 127 of the selected compartment 120, as disclosed herein.

Completing the intake operation at 640 may further comprise recording an intake record 562 within the data store 170 (and/or audit data 570), as disclosed herein. The intake record 562 may be recorded by the BPM 160 (and/or intake logic 162 thereof). The intake record 562 may comprise information pertaining to the intake operation, as disclosed herein, e.g., may indicate a date or time of the intake operation, identify personnel 103 responsible for the intake operation, identify the VU 105 loaded in the intake operation (by composite ID or the like), identify the quantity of VU 105 loaded in the intake operation, identify the compartment 120 and/or dispensing apparatus 110 into which the VU 105 were loaded, and so on. The intake logic 162 may be further configured to update inventory information maintained in the data store 170 at 640. Completing the intake operation may comprise updating the compartment record 520 of the selected compartment 120. At 640, the BPM 160 (and/or intake logic 162) may be configured to update the compartment record 520 of the selected compartment 120 to indicate the increased quantity of VU 105 loaded into the selected compartment 120 in the completed intake operation.

FIG. 6C is a flow diagram illustrating another example of a method 601 for managing an intake operation to load VU 105 into a dispensing apparatus 110, as disclosed herein. At 609, the intake operation may be initiated. The intake operation may be initiated in response to user input received through HMI resources 154 of the control module 140 (e.g., selection of an “intake” input or the like). Initiating the intake operation may comprise authenticating the identity of the HCP personnel 103 responsible for performing the intake operation. The identity of the personnel 103 may be authenticated by any suitable mechanism, as disclosed herein, e.g., password, biometric, multi-factor authentication, token, proximity (e.g., NFC), and/or the like.

At 610, a composite ID for the VU 105 being loaded in the intake operation may be acquired and, at 620, the BPM 160 (and/or intake logic 162 thereof) may select a compartment 120 to receive the VU 105 based on the composite ID at 620, as disclosed herein.

At 632, the control module 140 may be configured to provide access to the selected compartment 120 of the dispensing apparatus 110 while locking compartments 120 of the dispensing apparatus 110 other than the selected compartment 120 (and/or maintaining the other compartments 120 in a locked configuration). At 632, the control module 140 may be configured to cause the access device 122 of the selected compartment 120 to transition from the locked configuration to the unlocked configuration, as disclosed herein. The control module 140 may be further configured to cause access devices 122 of compartments 120 of the dispensing apparatus 110 other than the selected compartment 120 to transition to the locked configuration (and/or cause access devices 122 of the other compartments 120 to remain in the locked configuration).

At 632, the control module 140 may prevent human error in the intake operation. More specifically, while loading the VU 105, the control module 140 may unlock the selected compartment 120 while compartments 120 other than the selected compartment 120 are locked (and/or are maintained in locked configurations). Therefore, the personnel 103 responsible for performing the intake operation may be prevented from inadvertently loading the VU 105 into compartment(s) other than the selected compartment 120.

At 632, the control module 140 may be further configured to identify and/or indicate the selected compartment 120 to the personnel 103. The control module 140 may identify and/or indicate the selected compartment 120 by a) unlocking the selected compartment 120, b) causing a compartment HMI 124 of the selected compartment 120 to produce a visual output, c) causing a compartment HMI 124 of the selected compartment 120 to produce an audio output, d) causing an actuator 125 to at least partially open the selected compartment 120, and/or the like.

At 640, the intake operation may be completed, as disclosed herein.

FIG. 6D is a flow diagram of another example of a method 603 for managing the intake of beneficial products into a dispensing apparatus 110. At 611, the BPM 160 and/or inventory control logic 161 may be configured to initiate an intake operation for a VU 105 and acquire the composite ID of the VU 105, as disclosed herein (e.g., read a plurality of ID from the VU 105, including a first ID 131 and second ID 132).

Occupied compartments 120 matching the composite ID of the VU 105 may be identified at 622, as disclosed herein. If one or more occupied compartments 120 matching the composite ID are identified at 612, a matching, occupied compartment 120 may be selected at 623. In some implementations, the selecting at 622-623 may be based on hierarchical criteria, including a first criterion based on composite ID and a second criterion. The first criterion may be applied at 622 and the second criterion may be applied at 623. In other words, the first criterion may comprise identifying matching compartments 120 and the second criterion may select a compartment from the matching compartments 120 based on any suitable characteristic of the matching compartments 120, as disclosed herein. If a matching compartment 120 is selected at 623, the flow may continue at 631; otherwise, the flow may continue at 625.

At 631, the BPM 160 and/or inventory control logic 161 may prompt the operator to load the VU 105 into the occupied, matching compartment 120 identified at 623. The prompting may be implemented by use of HMI resources 154 and may comprise providing access to the selected compartment 120, as disclosed herein.

At 633, the BPM 160 and/or inventory control logic 161 may determine whether the VU was successfully loaded into the compartment 120 selected at 623. The determining may be based on user input received through the HMI resources 154, as disclosed herein. The determining may further comprise verifying that the VU 105 was loaded into the compartment 120 by use of, inter alia, one or more sensors 127, as disclosed herein. If the VU 105 was successfully loaded, the flow may continue at 640; otherwise, the flow may continue at 623, where the VU 105 may be loaded into a next matching compartment 120 (if any).

If the VU 105 cannot be loaded into a matching occupied compartment 120, the flow may continue at 634. At 634, the BPM 160 and/or inventory control logic 161 may be configured to identify empty compartments 120 of the dispensing apparatus 120. If empty compartments 120 are identified, the flow may continue at 635; otherwise, the flow may continue at 650.

At 635, the BPM 160 and/or inventory control logic 161 may select a compartment 120 from the empty compartments 120 identified at 634. The selection may be based on deterministic and/or random criteria. In some implementations, the selection may be based on an internal configuration 220 of the empty compartments 120. For example, the selection may proceed from smallest empty compartment 120 to largest empty compartment 120. In some examples, the selection may be based on hierarchical criteria, including a first criterion and second criterion. The first criterion may be based on a first characteristic, such as internal configuration 220, size, or the like. The second criterion may be based on another, different characteristic, such as physical proximity of the empty compartments 120 to other, occupied compartments 120, the MDC 112 of the empty compartments 120, a predetermined order of the empty compartments 120, and/or the like. Alternatively, the second criterion may be random (or pseudo random), as disclosed herein. If an empty compartment 120 is selected at 634-635, the flow may continue at 636; otherwise, the flow may continue at 652.

At 636, the BPM 160 and/or inventory control logic 161 may be configured to prompt the user to load the VU 105 into the empty compartment 120 selected at 634-635, which may comprise providing access to the selected empty compartment 120.

At 637, the BPM 160 and/or inventory control logic 161 may determine whether the VU 105 was successfully loaded into the selected empty compartment 120, as disclosed herein. If the VU 105 was successfully loaded, the flow may continue at 640; otherwise, the flow may continue at 635, where a next empty compartment 120 may be selected for the VU 105 (if any).

At 652, the VU 105 may be loaded into inventory in an alternative intake operation. The alternative intake operation may comprise storing the VU 105 within an alternative storage location, as disclosed herein.

At 640, the BPM 160 and/or inventory control logic 161 may complete the intake operation, as disclosed herein.

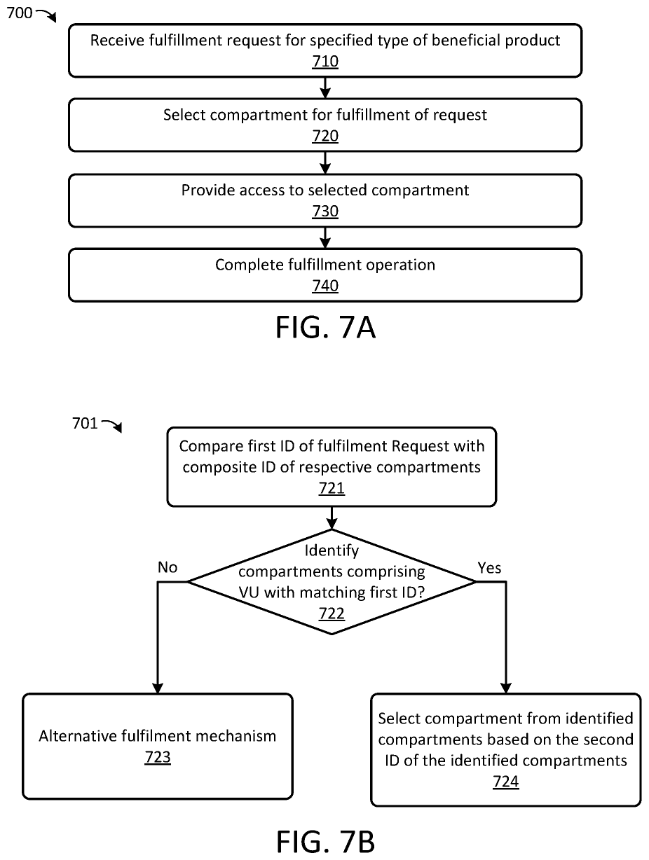

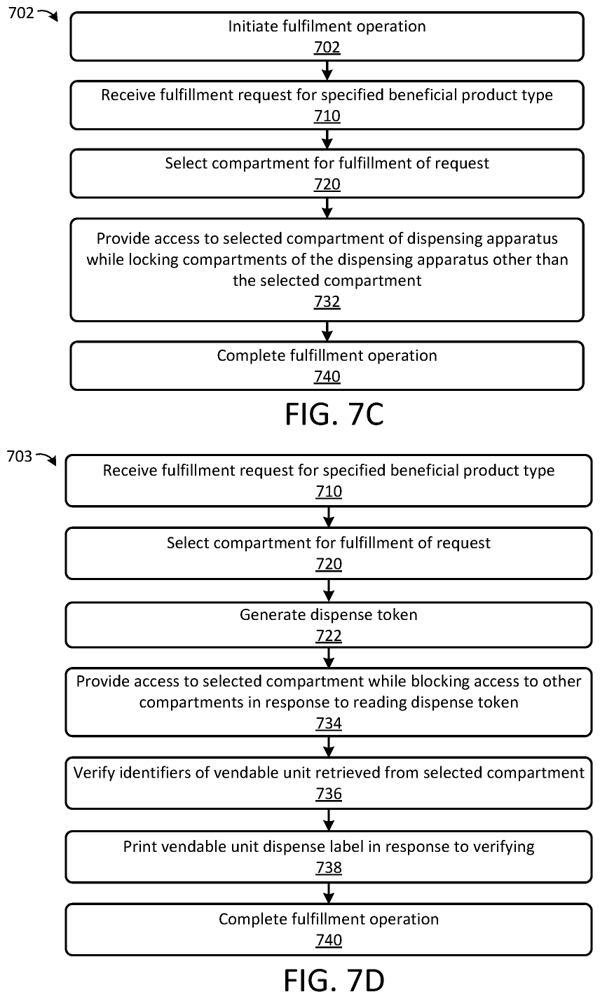

FIG. 7A is a flow diagram illustrating an example of a method 700 for managing a fulfilment operation to dispense a beneficial product to a patient 102. At 710, the BPM 160 may receive a fulfilment request. The fulfilment request may comprise a request to dispense a specified type of beneficial product to a patient 102. The fulfilment request may, for example, correspond to a prescription issued by authorized personnel 103 of the HCP 101, as disclosed herein. In some implementations, the fulfilment request may be recorded within the data store 170 (and/or audit data 570). For example, the fulfilment request of 710 may comprise and/or be embodied by a prescription record 505, as disclosed herein.

The fulfilment request of 710 may specify the type of beneficial product to be dispensed to the patient. The fulfilment request may a first ID 131 configured to specify the quantity, concentration, dosage, and/or other characteristics of the beneficial product. As disclosed herein, the first ID 131 may comprise an NDC, RxNorm, and/or other suitable ID determinative of beneficial product type.

At 720, the BPM 160 (and/or fulfilment logic 164 thereof) may select a compartment 120 for the fulfilment request. Selecting a compartment 120 for the fulfilment request may comprise selecting a compartment 120 that currently holds VU 105 comprising the type of beneficial products specified in the fulfilment request. More specifically, the BPM 160 may be configured to select a compartment 120 that comprises VU 105 having composite ID that match the first ID 131 (beneficial product type) of the fulfilment request. The selection of 720 may be implemented in accordance with method 701 illustrated in the flow chart of FIG. 7B.

At 721, the fulfilment logic 164 of the BPM 160 may be configured to compare the first ID 131 of the fulfilment request with composite ID of VU 105 held within respective compartments 120 of the dispensing apparatus 110. For example, the BPM 160 may search the data store 170 for compartment records 520 having a composite ID matching the first ID 131. The comparing at 721 may comprise identifying compartments 120 that currently hold VU 105 comprising beneficial products of the type specified in the fulfilment request.

At 722, the fulfilment logic 164 may determine whether compartments 120 comprising VU 105 that match the first ID 131 of the fulfilment request can be identified, e.g., are available at the dispensing apparatus 110. If compartments 120 are identified, the flow may continue at 723; otherwise, the flow may continue at 724.

At 723, the fulfilment logic 164 may determine that the type of beneficial product specified by the fulfilment request is not available at the dispensing apparatus 110 and, as such, may attempt to satisfy the fulfilment request by an alternative mechanism. The fulfilment logic 164 may, for example, attempt to satisfy the fulfilment request at another dispensing apparatus 110 of the HCP 101 (if any). The fulfilment logic 164 may, for example, search compartment records 520 of other dispensing apparatus 110 to identify compartments 120 comprising VU 105 having first ID 131 corresponding to the first ID 131 of the fulfilment request. Alternatively, or in addition, the fulfilment logic 164 may direct the patient 102 to fulfil the request at another external source, such as a retail pharmacy or the like.

At 724, the fulfilment logic 164 may select a compartment 120 from the compartments 120 identified at 721 and 722. The identified compartments 120 may comprise the type of beneficial product specified by the fulfilment request; since each of the identified compartments 120 comprises VU 105 matching the first ID 131 of the fulfilment request, each of the identified compartments 120 may comprise VU 105 suitable for satisfying the fulfilment request (e.g., each of the identified compartments 120 comprise the type of beneficial product specified by the fulfilment request).

The VU 105 held within the identified compartments 120 may have matching first ID 131 but may differ with respect to the second ID 132. In other words, each of the identified compartments 120 may comprise the same type of VU 105 (same type of beneficial product) from different respective lots or batches, per the second ID 132. Therefore, at 724, the fulfilment logic 164 may select a compartment 120 from the identified compartments 120 based on the second ID 132 of the identified compartments 120 (the aspect of the VU 105 that differs from compartment 120 to compartment 120).

The fulfilment logic 164 may select a compartment 120 from the identified compartments 120 in accordance with any suitable criterion and/or metric. In some implementations, the fulfilment logic 164 may utilize time-based selection criteria. For example, the selection may be based on expiration and/or use-by date. As disclosed herein, the second ID 132 may be determinative of the lot or batch of the beneficial product. The second ID 132 of a VU 105 may, therefore, correspond to an expiration or use-by date of the VU 105. At 723, the fulfilment logic 164 may be configured to select the compartment 120 having a second ID 132 corresponding to a soonest expiration and/or use-by date of the identified compartments 120. Although examples of selection criteria are described herein, the disclosure is not limited in this regard, and could be adapted to select between different lots or batches (second ID 132) in accordance with any suitable criteria or metric. For example, in some implementations, the costs associated with VU 105 may vary by second ID 132 and/or patient 102, e.g., due to health care coverage, provisions of patient health care plans, and/or the like. In these implementations, the selection of 723 may be based, at least in part, on costs associated with respective second ID 132.

Referring back to FIG. 7A, at 730, the control module 140 may provide access to the compartment 120 selected at 720. The control module 140 may provide access to the selected compartment 120 (e.g., may unlock the selected compartment 120). The control module 140 may be further configured to identify and/or indicate the selected compartment 120 (e.g., as described above in conjunction with FIGS. 6A and 6C). The control module 140 may, therefore, prevent human error in the fulfilment operation, as disclosed herein. The control module 140 may ensure that the VU 105 is dispensed from the correct, selected compartment 120, such that the VU 105 dispensed to the patient 102 has a known, accurate a) first ID 131 (beneficial product type) and b) second ID 132 (beneficial product lot or batch).

At 740, the fulfilment operation may be completed. Completing the fulfilment operation may comprise verifying that the VU 105 retrieved from the selected compartment 120 matches the composite ID expected for the VU 105 (e.g., matches the composite ID of the selected compartment 120). The verifying may comprise acquiring the composite ID of the VU 105 by use of the DAQ module 142 (e.g., interrogating the VU 105) and comparting the acquired composite ID with the composite ID of the selected compartment 120.

At 740, completing the fulfilment operation may further comprise outputting fulfilment data by use of the output module 144. The output module 144 may be configured to encode the fulfilment data on a DT 106, such as a tag, label, or the like. Completing the fulfilment operation may further include affixing the fulfilment data (e.g., DT 106) to the VU 105 in preparation for dispensing the VU 105 to the patient 102.

Completing the fulfilment operation may further comprise receiving, at the control module 140, an indication from personnel 103 that the fulfilment operation has been completed. The completion indication may be received through any suitable means, such as HMI resources 154 of the control module 140, compartment HMI 124 of the selected compartment 120, and/or the like. Alternatively, or in addition, the control module 140 may determine that the fulfilment operation is complete at 740 in response to verifying the composite ID of the VU 105, producing fulfilment data at the output module 144, detecting that the access device 122 of the selected compartment 120 has been returned to the closed (or locked) configuration, and/or the like.

Completing the fulfilment operation at 740 may further comprise recording a fulfilment record 564 within the data store 170 (and/or audit data 570), as disclosed herein. The fulfilment record 564 may be recorded by the BPM 160 (and/or fulfilment logic 164 thereof). The fulfilment record 564 may comprise information pertaining to the fulfilment operation, as disclosed herein, e.g., may indicate a date or time of the fulfilment operation, identify personnel 103 responsible for the fulfilment operation, identify the VU 105 dispensed in the fulfilment operation (by composite ID or the like), identify the compartment 120 and/or dispensing apparatus 110 from which the VU 105 was dispensed, identify the patient 102 to which the VU 105 was dispensed, reference the fulfilment request (e.g., prescription record 505), and so on. The fulfilment logic 164 may be further configured to update inventory information maintained in the data store 170 at 740. Completing the fulfilment operation may comprise updating the compartment record 520 of the selected compartment 120. At 740, the BPM 160 (and/or fulfilment logic 164) may be configured to update the compartment record 520 of the selected compartment 120 to indicate the decreased quantity of VU 105 available within the selected compartment 120 per the completed fulfilment operation. At 740, the BPM 160 may be further configured to update the prescription record 505 associated with the fulfilment request (if any) to indicate that the prescription has been fulfilled; for example, the BPM 160 may update the status of the prescription record 505 from “pending” to “completed” or “fulfilled.”

FIG. 7C is a flow diagram illustrating another example of a method 702 for managing a fulfilment operation at a dispensing apparatus 110. At 702, the fulfilment operation may be initiated. The fulfilment operation may be initiated in response to user input received through HMI resources 154 of the control module 140 (e.g., selection of an a “fulfilment” input or the like). Alternatively, or in addition, the fulfilment operation may be initiated in response to creation of a prescription by authorized personnel 103 of the HCP 101 and/or recording a corresponding prescription record 505 within the data store 170. In some implementations, the fulfilment operation may be initiated by a patient 102. The patient 102 may receive a prescription and may present the prescription (and/or token by which the corresponding prescription record 505 can be retrieved) to personnel 103 at the dispensing apparatus 110.

Initiating the fulfilment operation may further comprise authenticating the identity of the HCP personnel 103 responsible for performing the fulfilment operation. The identity of the personnel 103 may be authenticated by any suitable mechanism, as disclosed herein, e.g., password, biometric, multi-factor authentication, token, proximity (e.g., NFC), and/or the like.

At 710, the BPM 160 (and/or fulfilment logic 164) may receive a fulfilment request corresponding to the fulfilment operation. Receiving the fulfilment request may comprise retrieving a prescription record 505 from the data store 170 (and/or audit data 570). The fulfilment request may specify the type of beneficial product to be dispensed to the patient 102, as disclosed herein, e.g., may comprise a first ID 131 for the beneficial product. At 720, the BPM 160 (and/or fulfilment logic 164) may select a compartment 120 from which to dispense VU 105 to the patient 102 per the fulfilment request, as disclosed herein.

At 732, the control module 140 may be configured to provide access to the selected compartment 120 of the dispensing apparatus 110 while locking compartments 120 of the dispensing apparatus 110 other than the selected compartment 120 (and/or maintaining the other compartments 120 in a locked configuration). At 732, the control module 140 may be configured to cause the access device 122 of the selected compartment 120 to transition from the locked configuration to the unlocked configuration, as disclosed herein. The control module 140 may be further configured to cause access devices 122 of compartments 120 of the dispensing apparatus 110 other than the selected compartment 120 to transition to the locked configuration (and/or cause access devices 122 of the other compartments 120 to remain in the locked configuration).

At 732, the control module 140 may prevent human error in the fulfilment operation. More specifically, while retrieving VU 105 from the dispensing apparatus 110, the control module 140 may unlock the selected compartment 120 while compartments 120 other than the selected compartment 120 are locked (and/or are maintained in locked configurations). Therefore, the personnel 103 responsible for performing the fulfilment operation may be prevented from inadvertently retrieving VU 105 from a compartment other than the selected compartment 120. In other words, the personnel 103 may be prevented from retrieving VU 105 of the wrong type or lot. At 732, the control module 140 may be further configured to identify and/or indicate the selected compartment 120 to the personnel 103, as disclosed herein.

At 740, the provisioning operation may be completed, as disclosed herein.

FIG. 7D is a flow diagram illustrating a further example of a method 703 for managing a provisioning operation to dispense a specified type of beneficial product to a patient 102. At 710, the inventory control logic 161 may receive a fulfillment request to provision a specified type of beneficial product to a patient 102 and, at 720, the BPM 160 may be configured to select a compartment 120 of a dispensing apparatus 160 for the provisioning operation, as disclosed herein.

At 722, the BPM 160 may generate a dispense token 134 for the provisioning operation. The dispense token 134 may comprise a physical DT 135, a virtual DT 136, or the like. In some implementations the dispense token 134 may be generated at the selected dispensing apparatus 110. For example, the BPM 160 may configure the output module 142 of the dispensing apparatus 110 to print a physical DT 135. Alternatively, the dispense token 134 may be generated at an operator terminal 410. For example, the BPM 160 may configure a print device of the operator terminal 410 to produce a physical DT 135 configured to identify the selected dispensing station 110, compartment 120, VU 105 to be dispensed, the patient 102, and so on. Alternatively, or in addition, the BPM 160 may generate a virtual DT 136 for the provisioning operation. The virtual DT 136 may be configured for display and/or access through an application 137 of an operator computing device 138, as disclosed herein.

At 734, the BPM 160 may be configured to provide access to the selected compartment 120 in response to reading the dispense token 134, e.g., in response to the dispense token 134 being placed within a scan region of the DAQ module 142 of the dispensing apparatus 110. In response to reading the dispense token 134, the BPM 160 may be configured to provide access to the selected compartment 120 of the selected dispensing apparatus 110 while blocking access to other compartments 120 of the selected dispensing apparatus 110, as disclosed herein.

At 736, the BPM 160 may be configured to verify identifiers of the VU 105 retrieved from the selected compartment 120. The BPM 160 may be configured to read the first ID 131 and second ID 132 from the VU 105 and verify that the first ID 131 and second ID 132 correspond to the specified type of beneficial product stored within the selected compartment 120, e.g., per the first ID 131 and second ID 132 associated with the selected compartment 120 within the data store 170.

At 738, the BPM 160 may be configured to print a VU dispense label 534 in response to verifying the identifiers of the VU 105. The VU dispense label 534 may comprise an end-user label, e.g., may identify the patient 102, identify the beneficial product, indicate the dispense date of the VI 105, specify the expiration date of the beneficial product, and so on.

At 740, the BPM 160 may be configured to complete fulfillment of the provisioning operation, as disclosed herein. For example, the BPM 160 may be configured to record a fulfillment record 564 within the data store 170, the fulfillment record 564 configured to associate the patient 102 with the specified type of VU 105 dispensed thereto. The fulfillment record 564 may enable the system 100 to maintain associations between patients 102 and the types of beneficial products dispensed to the patients 102 (e.g., per the first ID 131 of the corresponding VU 105) and the lot or batch of the beneficial products (e.g., per the second ID 132 of the corresponding VU 105).

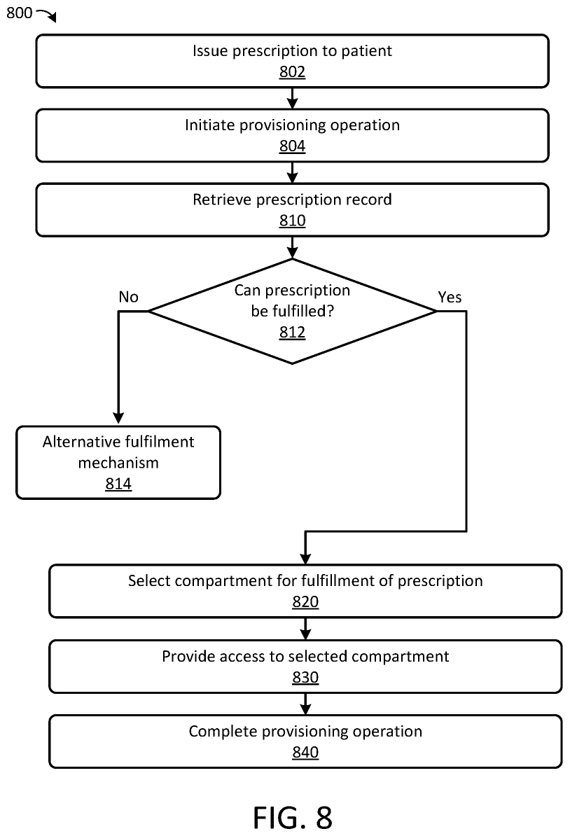

FIG. 8 is a flow diagram of a method 800 for dispensing beneficial products to patients 102, as disclosed herein.

At 802, authorized personnel 103 of an HCP 101, such as a doctor, may issue a prescription to a patient Issuing the prescription may comprise creating a prescription record 505 within the data store 170 (and/or audit data 570), as disclosed herein. The prescription record 505 may be configured to specify the of beneficial product prescribed to the patient (e.g., may comprise a first ID 131 configured to specify beneficial product type, quantity, concentration, dosage, and/or the like) The prescription record 505 may further comprise and/or reference patient authentication data (AUTH data), as disclosed herein. The prescription record 505 may further include status data. The status data may define parameters controlling fulfilment of the prescription, such as a time-to-live (TTL) for the prescription, a location perimeter within which the prescription may be fulfilled, may designate one or more dispensing apparatus 110 at which the prescription may be fulfilled, and/or the like. In some implementations, issuing the prescription may further comprise providing a tangible record of the prescription to the patient 102, which may comprise a physical token or record (e.g., receipt).

In some implementations, issuing the prescription may further comprise determining whether the dispensing apparatus 110 designated for use in fulfilment the prescription has sufficient inventory to fulfill the prescription. The inventory of the dispensing apparatus 110 may be determined as disclosed above in conjunction with FIGS. 7A-7C. If the designated dispensing apparatus 110 does not have sufficient inventory, an alternative dispensing apparatus 110 may be identified. Alternatively, fulfillment of the prescription may be delayed until inventory at the designated dispensing apparatus 110 becomes available (e.g., in an intake operation). Alternative, if the designated dispensing apparatus 110 has sufficient inventory, one or more 105 may be reserved for fulfilment of the prescription. Reserving the VU 105 may comprise marking the 105 as unavailable or “reserved” in the data store 170.

In some embodiments, issuing the prescription may further comprise indicating a payment amount for the prescription. The payment amount may represent the cost to the patient 102 to fulfill the prescription at a dispensing apparatus 110. The payment amount may not include health insurance and/or other benefits. Accordingly, the prescription may be fulfilled without adjudicating the prescription through health insurance adjudication and/or coordination of benefits programs. The payment amount may be set by the HCP 101, or other entity (e.g., the owner of the dispensing apparatus 110). Alternatively, the payment amount may reflect health insurance benefits of the patient 102. At 802, the patient may provide payment information pertaining to the prescription, such as credentialled payment information, which may be included in AUTH data of the prescription record 505.

At 804, a fulfillment or provisioning operation may be initiated at the control module 140 of the dispensing apparatus 110. Initiating the fulfilment operation may comprise receiving information pertaining to the prescription from the patient 102. For example, the fulfilment operation may be initiated in response to the patient 102 inputting AUTH data through HMI resources 154 of the control module 140.

Alternatively, or in addition, the fulfilment operation may be indicated in response to the patient 102 scanning a receipt or other tangible record of the prescription by use of the DAQ module 142, as disclosed herein.

At 810, the BPM 160 (and/or fulfilment logic 164 thereof) may retrieve a prescription record 505 from the data store 170 (and/or audit data 570). The prescription record 505 may be retrieved by use of the AUTH data, credentialed payment information, and/or other information provided by the patient 102 at 804.

At 812, the BPM 160 (and/or fulfilment logic 164) may determine whether the prescription can be fulfilled at the dispensing apparatus 110. The BPM 160 may evaluate status information of the prescription record 505, as disclosed herein. In a first non-limiting example, the BPM 160 may evaluate a TTL of the prescription to determine whether the patient 102 is attempting to fulfil the prescription within the time specified in the prescription record 505 (e.g., within the TTL). In a second non-limiting example, the BPM 160 may be configured to evaluate location restrictions of the prescription record 505 to determine, inter alia, whether the prescription is being fulfilled at or within the specified physical location or region. In a third non-limiting example, the BPM 160 may determine whether the patient 102 is attempting to fulfil the prescription at designated dispensing apparatus 110 (e.g., may determine whether the dispensing apparatus 110 at which the patient 102 presented the prescription is listed as a designated dispensing apparatus 110 in the prescription record 505). In a fourth non-limiting example, the BPM 160 may determine whether the prescription has already been fulfilled (e.g., may determine whether the status of the prescription record 505 has been updated to “completed” or “fulfilled” as disclosed herein). In a fifth non-limiting example, the BPM 160 may determine whether the dispensing apparatus 110 has sufficient inventory to fulfil the prescription, as disclosed herein. Although examples of fulfilment criteria are described herein, the disclosure is not limited in this regard and could be adapted to specify, evaluate, and/or enforce any suitable type of fulfilment restriction.

If the BPM 160 determines that the prescription can be filled at 812, the flow may continue at 820; otherwise, the flow may continue at 814.

At 814, the control module 140 may inform the patient 102 that the prescription cannot be fulfilled at the dispensing apparatus 110. The patient 102 may be inform through and/or by use of HMI resources 154 of the control module 140, as disclosed herein. In some implementations, the patient 102 may be informed as to the reason(s) that the prescription could not be fulfilled and/or may be directed to alternative fulfilment mechanisms, as disclosed herein.

The fulfilment logic 164 may select a compartment 120 for fulfilment of the prescription at 820, provide access to the selected compartment 120 at 830, and complete fulfilment of the prescription at 840, as disclosed herein.

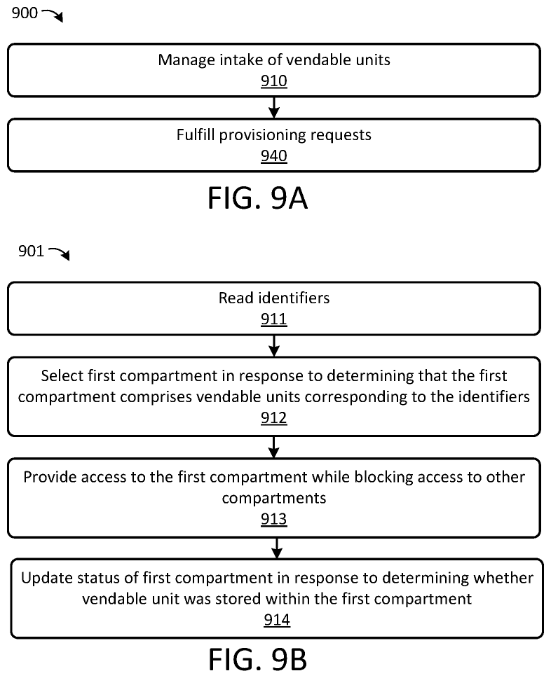

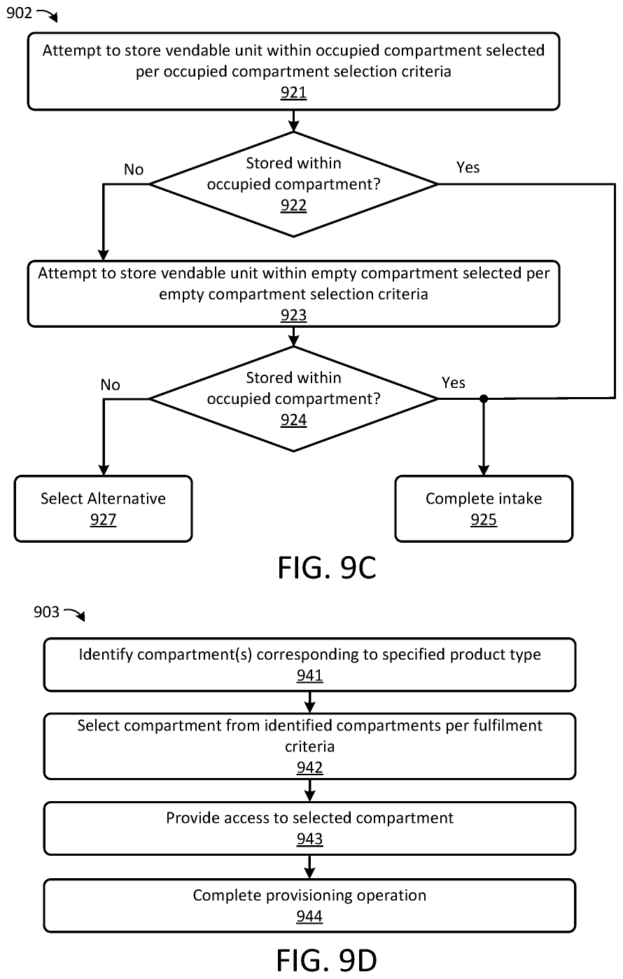

FIG. 9A is a flow diagram of another example of a method 900 for managing beneficial products. At 910, a BPM 160 and/or inventory control logic 161 may be configured to manage the intake of VU 105 to a dispensing apparatus 110. Managing the intake of the VU 105 may comprise a) determining a plurality of identifiers for respective VU 105, the plurality of identifiers comprising a first ID 131 determinative of product type and a second ID 132 different from the first ID 132 (e.g., a lot of batch ID), b) assigning VU 105 to compartments 120 of the dispensing apparatus 110, the assigning configured to group the VU 105 within the compartments 120 by both the first ID 131 and the second ID 132, and c) recording assignments between the VU 105 and the assigned compartments 120 within a data store 170.

In some embodiments, the intake logic may manage intake of a VU 105 as illustrated in method 901 of FIG. 9B. At 911, the inventory control logic 161 may be configured to read identifiers of the VU 105, as disclosed herein. The identifiers may comprise a first ID 131 and second ID 132, as disclosed herein. The inventory control logic 161 may be further configured to determine a composite ID for the VU 105. The identifiers may be read by use of a DAQ module 142 and/or acquisition device.

At 912, the inventory control logic 161 may be configured to select a first compartment 120 of the dispensing apparatus 120 to receive the VU 105.

In some implementations, selecting the first compartment 120 may comprise identifying one or more occupied compartments 120 of the dispensing apparatus 110 in response to determining that the identified compartments 120 comprise VU 105 having first ID 131 and second ID 132 corresponding to the first ID 131 and the second ID 132 of the vendable unit (and/or corresponding composite ID) and selecting the first compartment 120 from the one or more occupied compartments 120. The selection at 912 may be implemented by use of the data store 170, as disclosed herein (e.g., may comprise searching the data store 170 for a compartment record(s) 570 matching the identifiers of the VU 105). Alternatively, the first compartment 120 may be selected from one or more empty compartments 120 of the dispensing apparatus 110 in response to determining that none of the occupied compartments 120 of the dispensing apparatus 110 match the VU 105.

At 913, the inventory control logic 161 may be configured to provide access to the first compartment 120 while blocking access to compartments 120 of the dispensing apparatus 120 other than the first compartment 120. Providing access to a compartment 120 may comprise one or more of: unlocking the compartment 120, opening the compartment 120, displaying a visual prompt configured to distinguish the compartment 120 from other compartments 120 of the dispensing apparatus 110, and/or the like, as disclosed herein. Blocking access to a compartment may comprise one or more of: locking the compartment 120, closing the compartment 120, displaying a visual prompt configured to indicate that access to the compartment 120 is blocked, and/or the like, as disclosed herein.

At 914, the inventory control logic 161 may be configured to update a status of the first compartment 120 in the data store 170 in response to determining whether the VU 105 was successfully stored within the first compartment 120. In some implementations, the inventory control logic 161 may determine whether the VU 105 was successfully loaded in response to user input received through HMI resources 154. Alternatively, or in addition, the inventory control logic 161 may be configured to independently determine whether the VU 105 was loaded by use of one or more sensors 127, as disclosed herein.

The inventory control logic 161 may be configured to increase a quantity field of the corresponding compartment record 520 in response to determining that the VU 105 was successfully loaded into the first compartment 120. Alternatively, the inventory control logic 161 may be configured to set the full flag to “TRUE” and/or update the capacity field of the compartment record 520 in response to determining that the VU 105 could not be stored within the first compartment 120.

As disclosed herein, VU 105 may be assigned to compartments 120 such that the VU 105 are grouped per the hierarchical ID scheme 130. In other words, VU 105 may be stored within respective compartments 120 by composite ID (and/or by first ID and second ID). Therefore, each occupied compartment 120 of the dispensing apparatus 120 may comprise VU 105 having corresponding composite ID (and/or corresponding first ID and second ID).

In some implementations, VU 105 may be assigned to compartments 120 according to an assignment algorithm or scheme, as illustrated in method 902 of FIG. 9C. At 921, the inventory control logic 161 may be configured to attempt to store the VU 105 within an occupied compartment 120. The occupied compartment may be selected per occupied compartment selection (OCS) criteria. The OCS criteria may filter compartments 120 (and/or compartment records 520) by identifier. In other words, the OCS criteria may identify compartments 120 comprising corresponding VU 105, e.g., VU 105 having matching first ID 131 and second ID 132 (and/or matching composite ID). The OCS criteria may further select occupied compartments based on capacity, e.g., may exclude compartments 120 associated with a “TRUE” full flag and/or quantity field at (or within a threshold) of the determined capacity.

Attempting to store the VU 105 within an occupied compartment 120 may comprise a) identifying occupied compartments 120 that satisfy the OCS criteria (if any) b) selecting a first compartment 120 from the identified compartments 120, and c) attempting to load the VU 105 into the first compartment 120, e.g., provide access to the first compartment 120 while blocking access to compartments 120 other than the first compartment 120. If the VU 105 cannot be loaded into the first occupied compartment, the inventory control logic 161 may attempt to load the VU into a second compartment 120 selected from the occupied compartments 120 (if any), and so on.

At 922, the inventory control logic 161 may determine whether the VU 105 was successfully stored within an occupied compartment 120, as disclosed herein. If so, the flow may continue at 925; otherwise, the flow may continue at 923.

At 923, the inventory control logic may attempt to store the VU 105 within an empty compartment 120 of the dispensing apparatus 110 (if any). The inventory control logic 120 may be configured to select the empty compartment 120 per empty compartment selection (ECS) criteria. The ECS criteria may be based on any suitable characteristic. For example, the ECS criteria may select empty compartments 120 based on suitability rules. As disclosed herein, the interior volume 121 of a compartment 120 may be adapted to hold a particular type of VU 105, e.g., may comprise an internal element 128 with receptacles 129 configured to hold the particular type of VU 105. The ECS criteria may be configured to select empty compartments 120 based on configuration information defined within corresponding compartment records 520 maintained within the data store 170.

Alternatively, or in addition, the ECS criteria may be configured to select empty compartments 120 based on other characteristics, such as internal volume configuration (e.g., size of the internal volume 121 of the empty compartments 120). By way of non-limiting example, the ECS criteria may select empty compartments from smallest to largest. Per the ECS criteria, the inventory control logic 161 may initially select the smallest compartment 120 from the empty compartments 120, e.g., the first compartment 120 may correspond to a smallest compartment 120 of the empty compartments 120. If the VU 105 cannot be loaded into the smallest compartment 120, the inventory control logic 161 may select a second compartment 120 corresponding to a next larger compartment 120 of the empty compartments 120, and so on. In some implementations, the inventory control logic 161 may record information pertaining to respective types of VU 105, such as a smallest compartment size suitable for storage of the VU 105. Subsequent attempts to load the VU 105 may, therefore, select from the empty compartments 120 at the smallest suitable compartment size determined for the VU 105.

At 924, the inventory control logic may determine whether the VU 105 was successfully stored within an empty compartment 120. If so, the flow may continue at 925; otherwise, the flow may continue at 927.

At 925, the inventory control logic 161 may complete the intake operation, which may comprise updating the data store 170 to record an assignment between the VU 105 and the compartment 120 in which the VU 105 was stored, as disclosed herein. Alternatively, the update may be implemented at 922 and/or 924.

At 927, the inventory control logic 161 may select an alternative intake mechanism for the VU 105, as disclosed herein.

Referring back to FIG. 9A, the intake control logic 161 may be further configured to fulfil provisioning requests at the dispensing apparatus 110 at 940. Fulfilling a provisioning request for a specified product type may comprise identifying one or more compartments 120 assigned VU 105 having a first ID 131 corresponding to the specified product type and providing access to a designated compartment 120 of the identified compartments 120, the designated compartment 120 selected based on second ID 132 associated with the identified compartments 120.

In some implementations, the inventory control logic 161 may be configured to select compartments 120 from which to dispense VU 105 in accordance with defined fulfillment criteria. The inventory control logic 161 may implement provisioning operations in accordance with method 903 illustrated in FIG. 9D. More specifically, the inventory control logic 161 may implement aspects of the method 903 in response to a request to dispense a specified type of product at the dispensing apparatus 110. At 941, the inventory control logic 161 may be configured to identify compartment(s) 120 of the dispensing apparatus 10 that comprise VU 105 corresponding to the specified product type. The compartment(s) 120 may be identified by searching compartment records 520 of the data store 170, as disclosed herein.

At 942, the inventory control logic may be configured to select a compartment 120 from the identified compartments 120. The selection may be based on second ID 132 associated with the identified compartments 120. In some implementations, the selection may be implemented in accordance with defined fulfillment criteria. For example, the inventory control logic 161 may be configured to select compartments 120 from which to dispense VU 105 based on product expiration date (as opposed to intake date). The fulfilment criteria may correspond to a first-expire-first-out (FEFO) scheme, as disclosed herein (rather than a conventional FIFO scheme); at 942, the inventory control logic 161 may select the compartment 120 associated with a second ID 132 corresponding to an earliest or soonest expiration date of the compartments 120 identified at 941. In other words, the inventory control logic 161 may select a compartment 120 comprising VU 105 of the specified product type having the earliest expiration date.

At 943, the inventory control logic 943 may be configured to provide access to the selected compartment 120 and at 944 the inventory control logic 161 may be configured to complete the provisioning operation, as disclosed herein.

In some implementations, the inventory control logic 161 may be further configured to generate a dispense token 134 in response to selecting the compartment 120 for the fulfillment operation at 942. The dispense token 134 may identify the type of beneficial product and/or VU 105 to be dispensed from the selected compartment 120. The dispense token 134 may comprise a machine-readable tag or other indicia configured to be read by the DAQ module 142. An operator 103 may retrieve the dispense token 134, present the dispense token 134 at the DAQ module 142 and, in response to reading the physical token, the inventory control logic 161 may provide access to the selected compartment 120 at 943.

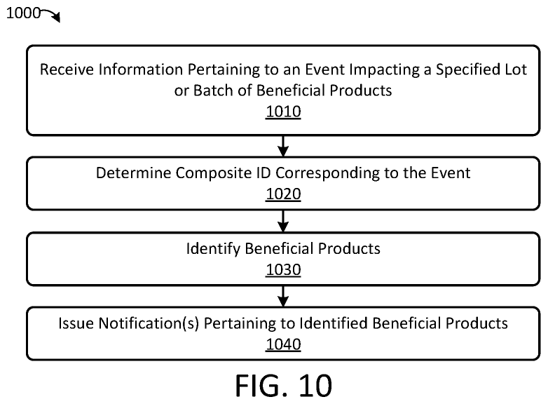

FIG. 10 is a flow diagram 1000 of a method for handling an event pertaining to a beneficial product. As used herein, an “event” pertaining to a beneficial product may comprise and/or refer to an event involving the management of beneficial products maintained within a dispensing apparatus 110 and/or dispensed to patients 102. An event may, for example, refer to a recall of a particular lot or batch of a beneficial product, expiration of the beneficial product, and/or the like.

At 1010, the BPM 160 may receive information pertaining to an event impacting a specified lot or batch of beneficial products. The event may pertain to a recall and/or expiration of the beneficial products. In some implementations, the information pertaining to the event may be received from an external entity, such as a government agency, e.g., the United States Food and Drug Administration (FDA), commercial entity, manufacturer, and/or the like. For example, the event may comprise a recall event issued by the FDA. Alternatively, or addition, information pertaining to the event may be determined by one or more components of the system 100. For example, the BPM 160 may be configured to record expiration and/or use-by dates for VU 105 maintained within dispensing apparatus 110 of the system 100. The BPM 160 may generate events corresponding to upcoming expiration and/or use-by dates of respective VU 105.

At 1020, the BPM 160 may determine a composite ID corresponding to the event, e.g., determine the composite ID of beneficial products impacted by the event. The events disclosed herein may impact specified lots or batches of a specified type of beneficial product. Accordingly, the information received at 1010 may specify the a) type and b) lot or batch of beneficial products impacted by the event. The type of beneficial product impacted by the event may correspond to a first ID 131 of the hierarchical ID scheme 130 (e.g., NDC, RxNorm, and/or the like). The lot or batch of beneficial products impacted by the event may correspond to the second ID 132 of the hierarchical ID scheme 130 (e.g., may comprise a lot ID, such as a lot number, batch number, and/or the like).

At 1030, the BPM 160 may identify beneficial products impacted by the event. The BPM 160 may identify impacted beneficial products a) within the inventory of the HCP 101 and/or b) impacted beneficial products dispensed to patients 102 of the HCP 101.

Impacted beneficial products within the inventory of the HCP 101 may be identified by, inter alia, searching compartment records 520 of the data store 170. The compartment records 520 may be searched to identify compartment records 520 having composite ID matching the composite ID determined at 1020.

Impacted beneficial products dispensed to patients 102 may be identified by, inter alia, searching the data store 170 and/or audit data 570. For example, the BPM 160 may search the data store 170 (and/or audit data 570) for fulfilment records 564 comprising composite ID matching the composite ID determined at 1020. Alternatively, or in addition, the BPM 160 may search the patient audit data 572 for patient audit records 574 matching the composite ID.

At 1040, the BPM 160 may be configured to issue notifications pertaining to the beneficial products identified at 1030. The BPM 160 may issue internal, HCP notifications configured to notify HCP personnel 103 of impacted beneficial products currently being held within the inventory of the HCP 101. The HCP notifications may identify the specific compartments 120 in which the impacted beneficial products (e.g., VU 105) are being held, which may enable HCP personnel 103 to efficiently remove the impacted beneficial products from the inventory, while minimizing the potential for human error. Since the disclosed BPM 160 (and/or control module 140) is configured to group VU 105 within respective compartments 120 by a combination of a) beneficial product type (first ID 131) and b) beneficial product lot or batch (second ID 132), the impacted beneficial products can be removed from inventory by purging the contents of identified compartments 120, without the need to manually inspecting each VU 105.

The BPM may be further configured to issue external, patient notifications. The patient notifications may be issued to patients 102 to which impacted beneficial products were dispensed (e.g., patients 102 identified at 1030). The patient notifications may identify the specific VU 105 issued to the patients 102. The patients 102 impacted by the event may be accurately identified due to, inter alia, prevention of human error during intake and fulfilment operations, as disclosed herein. Moreover, the patients 102 impacted by the event may be identified quickly and efficiently by use of the data maintained in the data store 170, e.g., granular fulfillment records 564, patient audit records 574, and so on.

This disclosure has been made with reference to various example implementations and/or embodiments. However, those skilled in the art will recognize that changes and modifications may be made to the examples described herein without departing from the scope of the disclosure. For example, various operational steps, as well as components for carrying out operational steps, may be implemented in alternate ways depending upon the particular application or in consideration of any number of cost functions associated with the operation of the system, e.g., one or more of the steps may be deleted, modified, or combined with other steps.

Additionally, as will be appreciated by one of ordinary skill in the art, principles of the present disclosure may be reflected in a computer program product on a computer-readable storage medium having computer-readable program code means embodied in the storage medium. Any tangible, non-transitory computer-readable storage medium may be utilized, including magnetic storage devices (hard disks, floppy disks, and the like), optical storage devices (CD-ROMs, DVDs, Blu-Ray discs, and the like), flash memory, and/or the like. These computer program instructions may be loaded onto a general-purpose computer, special purpose computer, or other programmable data processing apparatus to produce a machine, such that the instructions that execute on the computer or other programmable data processing apparatus create means for implementing the functions specified. These computer program instructions may also be stored in a computer-readable memory that can direct a computer or other programmable data processing apparatus to function in a particular manner, such that the instructions stored in the computer-readable memory produce an article of manufacture, including implementing means that implement the function specified. The computer program instructions may also be loaded onto a computer or other programmable data processing apparatus to cause a series of operational steps to be performed on the computer or other programmable apparatus to produce a computer-implemented process, such that the instructions that execute on the computer or other programmable apparatus provide steps for implementing the functions specified.

While the principles of this disclosure have been shown in various embodiments, many modifications of structure, arrangements, proportions, elements, materials, and components, which are particularly adapted for a specific environment and operating requirements, may be used without departing from the principles and scope of this disclosure. These and other changes or modifications are intended to be included within the scope of the present disclosure.

The foregoing specification has been described with reference to various embodiments. However, one of ordinary skill in the art will appreciate that various modifications and changes can be made without departing from the scope of the present disclosure. Accordingly, this disclosure is to be regarded in an illustrative rather than a restrictive sense, and all such modifications are intended to be included within the scope thereof. Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. As used herein, the terms “comprises,” “comprising,” and any other variation thereof, are intended to cover a non-exclusive inclusion, such that a process, a method, an article, or an apparatus that comprises a list of elements does not include only those elements but may include other elements not expressly listed or inherent to such process, method, system, article, or apparatus. Also, as used herein, the terms “coupled,” “coupling,” and any other variation thereof are intended to cover a physical connection, an electrical connection, a magnetic connection, an optical connection, a communicative connection, a functional connection, and/or any other connection.

Those having skill in the art will appreciate that many changes may be made to the details of the above-described embodiments without departing from the underlying principles of the invention. The scope of the present invention should, therefore, be determined only by the claims.

We claim:

1. A method for managing beneficial products, comprising:
managing the intake of vendable units into a dispensing apparatus, comprising:
determining a plurality of identifiers for respective vendable units, the plurality of identifiers comprising a first identifier determinative of product type and a second identifier different from the first identifier,
assigning vendable units to compartments of the dispensing apparatus, the assigning configured to group the vendable units within the compartments by both the first identifier and the second identifier,
recording assignments between the vendable units and the assigned compartments within a data store; and
fulfilling provisioning requests at the dispensing apparatus, wherein fulfilling a provisioning request for a specified product type comprises:
identifying one or more compartments assigned vendable units having a first identifier corresponding to the specified product type, and
providing access to a designated compartment of the identified compartments, the designated compartment selected based on second identifiers associated with the identified compartments.

2. The method of claim 1, wherein assigning a vendable unit to a compartment of the dispensing apparatus comprises:
reading the first identifier and the second identifier from the vendable unit,
wherein the first identifier comprises one or more of a universal product code, a national drug code, a Global Trade Identification Number, and an RxNorm identifier, and
wherein the second identifier comprises one or more of a lot identifier and a batch identifier.

3. The method of claim 2, wherein assigning the vendable unit to the compartment of the dispensing apparatus further comprises:
selecting a first compartment of the dispensing apparatus to receive the vendable unit;
providing access to the first compartment while blocking access to other compartments of the dispensing apparatus; and
updating a status of the first compartment in the data store in response to determining whether the vendable unit was successfully stored within the first compartment.

4. The method of claim 3, wherein:
providing access to the first compartment comprises one or more of: unlocking the first compartment, opening the first compartment, and displaying a first visual prompt configured to distinguish the first compartment from the other compartments; and
blocking access to the other compartments of the dispensing apparatus comprises one or more of: locking the other compartments, closing the other compartments, and displaying a second visual prompt configured to indicate that access to the other compartments is blocked.

5. The method of claim 3, wherein assigning the vendable unit to the compartment of the dispensing apparatus further comprises:
identifying one or more occupied compartments in response to determining that the identified compartments comprise vendable units having first identifiers and second identifiers corresponding to the first identifier and the second identifier of the vendable unit; and
selecting the first compartment from the one or more occupied compartments.

6. The method of claim 5, wherein assigning the vendable unit to the compartment of the dispensing apparatus further comprises:
updating the data store to indicate that the first compartment is fully occupied in response to determining that the vendable unit could not be stored within the first compartment;
selecting a second compartment from the one or more occupied compartments; and
providing access to the second compartment while blocking access to compartments of the dispensing apparatus other than the second compartment.

7. The method of claim 3, wherein assigning the vendable unit to the compartment of the dispensing apparatus further comprises:
identifying one or more empty compartments of the dispensing apparatus;
selecting the first compartment from the one or more empty compartments based, at least in part, on respective sizes of the one or more empty compartments.

8. The method of claim 7, wherein assigning the vendable unit to the compartment of the dispensing apparatus further comprises:
selecting a second compartment from the empty compartments in response to determining that the vendable unit could not be stored within the first compartment, the second compartment selected in response to determining that a size of the second compartment is larger than a size of the first compartment; and
providing access to the second compartment while blocking access to compartments of the dispensing apparatus other than the second compartment; and
updating the data store to indicate that the vendable unit is assigned to the second compartment.

9. The method of claim 1, wherein the second identifier is indicative of product expiration date, and wherein fulfilling the provisioning request further comprises selecting the designated compartment of the identified compartments based, at least in part, on product expiration dates associated with vendable units stored within respective compartments of the identified compartments.

10. The method of claim 9, wherein fulfilling the provisioning request further comprises one or more of:
printing a physical token comprising a computer-readable code identifying the designated compartment; and
providing access to the designated compartment while blocking access to compartments of the dispensing apparatus other than the designated compartment,
wherein providing access comprises one or more of: unlocking the designated compartment, opening the designated compartment, and displaying a visual indicator configured to distinguish the designated compartment from other compartments of the dispensing apparatus, and
wherein blocking access to the other compartments of the dispensing apparatus comprises one or more of: locking the other compartments, closing the other compartments, and displaying a second visual prompt configured to indicate that access to the other compartments is blocked.

11. A system for managing beneficial products, comprising:
a dispensing apparatus comprising a plurality of compartments; and
inventory control logic configured for operation on a processor, the inventory control logic configured to implement intake operations to load vendable units into the dispensing apparatus, the intake operations comprising:
determining a plurality of identifiers for respective vendable units, comprising a first identifier and a second identifier different from the first identifier, wherein the first identifier comprises one or more of a universal product code, a national drug code, a Global Trade Identification Number, and an RxNorm identifier,
assigning the vendable units to compartments of the dispensing apparatus such that the vendable units are grouped within the compartments by both the first identifier and the second identifier, and
recording assignments between the vendable units and the assigned compartments within a data store; and
wherein the inventory control logic is further configured to implement provisioning operations to dispense vendable units from the dispensing apparatus, and wherein implementing a provisioning operation to dispense a specified product type comprises:
identifying one or more compartments assigned vendable units having a first identifier corresponding to the specified product type, and
providing access to a designated compartment of the identified compartments, the designated compartment selected based on second identifiers associated with the identified compartments.

12. The system of claim 11, further comprising an acquisition device communicatively coupled to the inventory control logic, the acquisition device configured to read one or more of the first identifier and the second identifier from respective vendable units;
wherein, to assign a vendable unit to a compartment of the dispensing apparatus, the inventory control logic is configured to:
select a first compartment of the dispensing apparatus in response to determining that the first compartment comprises vendable units corresponding to the first identifier and the second identifier of the vendable unit,
provide access to the first compartment while blocking access to other compartments of the dispensing apparatus, and
update a status of the first compartment in a data store in response to determining whether the vendable unit was successfully stored within the first compartment;
wherein providing access to the first compartment comprises one or more of: unlocking the first compartment, opening the first compartment, and displaying a first visual prompt configured to distinguish the first compartment from the other compartments; and
wherein blocking access to the other compartments of the dispensing apparatus comprises one or more of: locking the other compartments, closing the other compartments, and displaying a second visual prompt configured to indicate that access to the other compartments is blocked.

13. The system of claim 12, wherein, to assign the vendable unit to the compartment of the dispensing apparatus, the inventory control logic is further configured to:
identify one or more occupied compartments in response to determining that the identified compartments comprise vendable units having first identifiers and second identifiers corresponding to the first identifier and the second identifier of the vendable unit; and
select the first compartment from the one or more occupied compartments.

14. The system of claim 13, wherein, to assign the vendable unit to the compartment of the dispensing apparatus, the inventory control logic is further configured to:
update the data store to indicate that the first compartment is fully occupied in response to determining that the vendable unit could not be stored within the first compartment;
select a second compartment from the one or more occupied compartments; and
provide access to the second compartment while blocking access to compartments of the dispensing apparatus other than the second compartment.

15. The system of claim 13, wherein, to assign the vendable unit to the compartment of the dispensing apparatus, the inventory control logic is further configured to:
identify one or more empty compartments of the dispensing apparatus;
select the first compartment from the one or more empty compartments based, at least in part, on respective sizes of the one or more empty compartments.

16. The system of claim 15, wherein, to assign the vendable unit to the compartment of the dispensing apparatus, the inventory control logic is further configured to:
select a second compartment from the empty compartments in response to determining that the vendable unit could not be stored within the first compartment, the second compartment selected in response to determining that a size of the second compartment is larger than a size of the first compartment; and
provide access to the second compartment while blocking access to compartments of the dispensing apparatus other than the second compartment; and
update the data store to indicate that the vendable unit is assigned to the second compartment.

17. The system of claim 11, wherein the second identifier is indicative of product expiration date, and wherein, to implement the provisioning operation to dispense the specified product type, the inventory control logic is further configured to:
select the designated compartment of the identified compartments based, at least in part, on product expiration dates associated with vendable units stored within respective compartments of the identified compartments.

18. The system of claim 17, further comprising a print device, wherein, to implement the provisioning operation, the inventory control logic is further configured to:
print a physical token comprising a computer-readable code identifying the selected compartment by use of the print device; and
provide access to the selected compartment while blocking access to compartments of the dispensing apparatus other than the designated compartment,
wherein providing access comprises one or more of: unlocking the selected compartment, opening the selected compartment, and displaying a visual indicator configured to distinguish the selected compartment from other compartments of the dispensing apparatus, and
wherein blocking access to the other compartments of the dispensing apparatus comprises one or more of: locking the other compartments, closing the other compartments, and displaying a second visual prompt configured to indicate that access to the other compartments is blocked.

19. A non-transitory computer-readable storage medium comprising instructions configured to cause a processor to implement operations for dispensing beneficial products, the operations comprising:
managing the intake of vendable units into a dispensing apparatus, comprising:
determining composite identifiers for respective vendable units, the composite identifiers comprising at least a portion of a first identifier determinative of product type and a second identifier different from the first identifier, wherein the first identifier comprises one or more of a universal product code, a national drug code, a Global Trade Identification Number, and an RxNorm identifier, and wherein the second identifier corresponds to product expiration date,
assigning vendable units to compartments of the dispensing apparatus, the assigning configured to group the vendable units within the compartments by composite identifier,
recording assignments between the vendable units and the assigned compartments within a data store; and
fulfilling provisioning requests at the dispensing apparatus, wherein fulfilling a provisioning request for a specified product type comprises:
identifying one or more compartments assigned vendable units having a composite identifier corresponding to the specified product type,
selecting a compartment from the one or more identified compartments based on expiration dates indicated by the second identifiers associated with respective identified compartments, and
providing access the selected compartment,
wherein assigning a vendable unit to a compartment of the dispensing apparatus comprises:
reading the first identifier and the second identifier from the vendable unit,
selecting a first compartment of the dispensing apparatus in response to determining that the first compartment comprises vendable units corresponding to the composite identifier of the vendable unit,
providing access to the first compartment while blocking access to other compartments of the dispensing apparatus, and
updating a status of the first compartment in the data store in response to user input indicating whether the vendable unit was successfully stored within the first compartment;
wherein providing access to a designated compartment of the dispensing apparatus comprises one or more of: unlocking the designated compartment, opening the designated compartment, and displaying a first visual prompt configured to distinguish the designated compartment from the other compartments; and
wherein blocking access to the other compartments of the dispensing apparatus comprises one or more of: locking the other compartments, closing the other compartments, and displaying a second visual prompt configured to indicate that access to the other compartments is blocked.

20. The non-transitory computer-readable storage medium of claim 19, wherein assigning the vendable unit to the compartment of the dispensing apparatus further comprises:
identifying one or more occupied compartments in response to determining that the identified compartments comprise vendable units having composite identifiers corresponding to the composite identifier of the vendable unit;
selecting the first compartment from the one or more occupied compartments; and
assigning a second compartment to the vendable unit in response to determining that the vendable unit could not be stored within the first compartment, the second compartment comprising one or more of:
a compartment selected from the one or more occupied compartments, and
an empty compartment of the dispensing apparatus.
